SEQUENCE LISTING (65 sequences)

SEQ ID NO: 1 - protein, 132 aa, Bacteriophage Q-beta
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 2 - protein, 329 aa, Bacteriophage Q-beta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYWTLLIAGGGSGSKPDPVIPDPPIDPPPGTGKYTCPFAIWSLEEVYEPPTKNRPWPIYNAVELQPREFDVALKDLLGNTKWRDWDSRLSYTTFRGCRGNGYIDLDATYLATDQAMRDQKYDIREGKKPGAFGNIERFIYLKSINAYCSLSDIAAYHADGVIVGFWRDPSSGGAIPFDFTKFDKTKCPIQAVIVVPRA

SEQ ID NO: 3 - protein, 129 aa, Bacteriophage R17
ASNFTQFVLVNDGGTGNVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 4 - protein, 130 aa, Bacteriophage fr
MASNFEEFVLVDNGGTGDVKVAPSNFANGVAEWISSNSRSQAYKVTCSVRQSSANNRKYTVKVEVPKVATQVQGGVELPVAAWRSYMNMELTIPVFATNDDCALIVKALQGTFKTGNPIATAIAANSGIY

SEQ ID NO: 5 - protein, 130 aa, Bacteriophage GA
MATLRSFVLVDNGGTGNVTVVPVSNANGVAEWLSNNSRSQAYRVTASYRASGADKRKYAIKLEVPKIVTQVVNGVELPGSAWKAYASIDLTIPIFAATDDVTVISKSLAGLFKVGNPIAEAISSQSGFYA

SEQ ID NO: 6 - protein, 132 aa, Bacteriophage SP
MAKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAY

SEQ ID NO: 7 - protein, 329 aa, Bacteriophage SP
AKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAYWAALLVASSGGGDNPSDPDVPVVPDVKPPDGTGRYKCPFACYRLGSIYEVGKEGSPDIYERGDEVSVTFDYALEDFLGNTNWRNWDQRLSDYDIANRRRCRGNGYIDLDATAMQSDDFVLSGRYGVRKVKFPGAFGSIKYLLNIQGDAWLDLSEVTAYRSYGMVIGFWTDSKSPQLPTDFTQFNSANCPVQTVIIIPS

SEQ ID NO: 8 - protein, 130 aa, Bacteriophage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 9 - protein, 133 aa, Bacteriophage M11
MAKLQAITLSGIGKKGDVTLDLNPRGVNPTNGVAALSEAGAVPALEKRVTISVSQPSRNRKNYKVQVKIQNPTSCTASGTCDPSVTRSAYSDVTFSFTQYSTVEERALVRTELQALLADPMLVNAIDNLNPAY

SEQ ID NO: 10 - protein, 133 aa, Bacteriophage MX1
MAKLQAITLSGIGKNGDVTLNLNPRGVNPTNGVAALSEAGAVPALEKRVTISVSQPSRNRKNYKVQVKIQNPTSCTASGTCDPSVTRSAYADVTFSFTQYSTDEERALVRTELKALLADPMLIDAIDNLNPAY

SEQ ID NO: 11 - protein, 330 aa, Bacteriophage NL95
MAKLNKVTLTGIGKAGNQTLTLTPRGVNPTNGVASLSEAGAVPALEKRVTVSVAQPSRNRKNYKVQIKLQNPTACTKDACDPSVTRSGSRDVTLSFTSYSTERERALIRTELAALLKDDLIVDAIDNLNPAYWAALLAASPGGGNNPYPGVPDSPNVKPPGGTGTYRCPFACYRRGELITEAKDGACALYACGSEALVEFEYALEDFLGNEFWRNWDGRLSKYDIETHRRCRGNGYVDLDASVMQSDEYVLSGAYDVVKMQPPGTFDSPRYYLHLMDGIYVDLAEVTAYRSYGMVIGFWTDSKSPQLPTDFTRFNRHNCPVQTVIVIPSL

SEQ ID NO: 12 - protein, 129 aa, Bacteriophage f2
ASNFTQFVLVNDGGTGNVTVAPSNFANGVAEWISSNSRSQAYKVTCSVRQSSAQNRKYTIKVEVPKVATQTVGGVELPVAAWRSYLNLELTIPIFATNSDCELIVKAMQGLLKDGNPIPSAIAANSGIY

SEQ ID NO: 13 - protein, 128 aa, Bacteriophage PP7
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 14 - protein, 131 aa, bacteriophage AP205
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 15 - protein, 132 aa, Artificial Sequence
  Bacteriophage Qbeta 240 mutant
AKLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 16 - protein, 132 aa, Artificial Sequence
  Bacteriophage Q-beta 243 mutant
AKLETVTLGKIGKDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 17 - protein, 132 aa, Artificial Sequence
  Bacteriophage Q-beta 250 mutant
ARLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 18 - protein, 132 aa, Artificial Sequence
  Bacteriophage Q-beta 251 mutant
AKLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 19 - protein, 132 aa, Artificial Sequence
  Bacteriophage Q-beta 259 mutant
ARLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 20 - protein, 185 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 21 - protein, 188 aa, artificial sequence
  Modified Hepatitis B virus
MDIDPYKEFGATVELLSFLPSDFFPSVRDLLDTAAALYRDALESPEHCSPHHTALRQAILCWGDLMTLATWVGTNLEDGGKGGSRDLVVSYVNTNVGLKFRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 22 - protein, 9 aa, Mus musculus
RPPGFSPFR

SEQ ID NO: 23 - protein, 8 aa, Mus musculus
RPPGFSPF

SEQ ID NO: 24 - protein, 352 aa, Homo sapiens [sequence omitted]

SEQ ID NO: 25 - protein, 10 aa, artificial sequence
  Synthetic CCR5 ECL2A
RSQKEGLHYT

SEQ ID NO: 26 - protein, 12 aa, artificial sequence
  Synthetic CCR5 ECL2A cyclic
CRSQKEGLHYTG

SEQ ID NO: 27 - protein, 31 aa, artificial sequence
  synthetic CCR5 PNt
MDYQVSSPIYDINYYTSEPCQKINVKQIAAR

SEQ ID NO: 28 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 29 - protein, 10 aa, artificial sequence
  synthetic CXCR4 extracellular ECL2 domain fragment
NVSEADDRYI

SEQ ID NO: 30 - protein, 39 aa, artificial sequence
  synthetic CXCR4 extracellular ECL2 domain fragment
MEGISIYTSDNYTEEMGSGDYDSMKEPCFREENANFNKI

SEQ ID NO: 31 - protein, 476 aa, Homo sapiens
CSKGTSHEAGIVCRITKPALLVLNHETAKVIQTAFQRASYPDITGEKAMMLLGQVKYGLHNIQISHLSIASSQVELVEAKSIDVSIQNVSVVFKGTLKYGYTTAWWLGIDQSIDFEIDSAIDLQINTQLTCDSGRVRTDAPDCYLSFHKLLLHLQGEREPGWIKQLFTNFISFTLKLVLKGQICKEINVISNIMADFVQTRAASILSDGDIGVDISLTGDPVITASYLESHHKGHFIYKNVSEDLPLPTFSPTLLGDSRMLYFWFSERVFHSLAKVAFQDGRLMLSLMGDEFKAVLETWGFNTNQEIFQEVVGGFPSQAQVTVHCLKMPKISCQNKGVVVNSSVMVKFLFPRPDQQHSVAYTFEEDIVTTVQASYSKKKLFLSLLDFQITPKTVSNLTESSSESIQSFLQSMITAVGIPEVMSRLEVVFTALMNSKGVSLFDIINPEIITRDGFLLLQMDFGFPEHLLVDFLQSLS

SEQ ID NO: 32 - protein, 16 aa, artificial sequence
  synthetic CETP fragment
FGFPEHLLVDFLQSLS

SEQ ID NO: 33 - protein, 9 aa, artificial sequence
  Modified human gastrin
EGPWLEEEE

SEQ ID NO: 34 - protein, 17 aa, Homo sapiens
EGPWLEEEEEAYGWMDF

SEQ ID NO: 35 - protein, 34 aa, homo sapiens
ELGPQGPPHLVADPSKKQGPWLEEEEEAYGWMDF

SEQ ID NO: 36 - protein, 18 aa, Homo sapiens
EGPWLEEEEEAYGWMDFG

SEQ ID NO: 37 - protein, 35 aa, Homo sapiens
ELGPQGPPHLVADPSKKQGPWLEEEEEAYGWMDFG

SEQ ID NO: 38 - protein, 21 aa, artificial sequence
  Modified human gastrin with linker sequence
CGGQGPWLEEEEEAYGWMDFG

SEQ ID NO: 39 - protein, 16 aa, artificial sequence
  Modified human gastrin with linker sequence
EGPWLEEEESSPPPPC

SEQ ID NO: 40 - protein, 20 aa, artificial sequence
  Modified human gastrin 1-17GGC
EGPWLEEEEEAYGWMDFGGC

SEQ ID NO: 41 - protein, 20 aa, artificial sequence
  Modified human gastrin CGG1-17amide
CGGQGPWLEEEEEAYGWMDF

SEQ ID NO: 42 - protein, 37 aa, artificial sequence
  Modified human gastrin CGG1-34amide
CGGQLGPQGPPHLVADPSKKQGPWLEEEEEAYGWMDF

SEQ ID NO: 43 - protein, 38 aa, artificial sequence
  Modified human gastrin 1-34G
CGGQLGPQGPPHLVADPSKKQGPWLEEEEEAYGWMDFG

SEQ ID NO: 44 - protein, 32 aa, artificial sequence
  synthetic CCR5 PNt-CC
MDYQVSSPIYDINYYTSEPCQKINVKQIAARC

SEQ ID NO: 45 - protein, 74 aa, Homo sapiens
TLQKKIEEIAAKYKHSVVKKCCYDGACVNNDETCEQRAARISLGPRCIKAFTECCVVASQLRANISHKDMQLGR

SEQ ID NO: 46 - protein, 20 aa, artificial sequence
  synthetic C5a 55-74
CVVASQLRANISHKDMQLGR

SEQ ID NO: 47 - protein, 82 aa, artificial sequence
  modified mouse CGSGG C5a
CGSGGNLHLLRQKIEEQAAKYKHSVPKKCCYDGARVNFYETCEERVARVTIGPLCIRAFNECCTIANKIRKESPHKPVQLGR

SEQ ID NO: 48 - protein, 22 aa, artificial sequence
  modified mouse CGGC5A59-77
CGGTIANKIRKESPHKPVQLGR

SEQ ID NO: 49 - protein, 12 aa, artificial sequence
  synthetic cyclic CXCR4 extracellular ECL2 domain fragment
CNVSEADDRYIG

SEQ ID NO: 50 - protein, 10 aa, Homo sapiens
KRPPGFSPFR

SEQ ID NO: 51 - protein, 9 aa, Homo sapiens
KRPPGFSPF

SEQ ID NO: 52 - protein, 12 aa, artificial sequence
  synthetic cyclized ECL2A
GRSQKEGLHYTC

SEQ ID NO: 53 - protein, 12 aa, artificial sequence
  synthetic CXCR4 extracellular ECL2 domain fragment
GNVSEADDRYIC

SEQ ID NO: 54 - protein, 32 aa, artificial sequence
  synthetic CCR5 PNt domain with C20 to Serine and C fused at the C terminus
MDYQVSSPIYDINYYTSEPSQKINVKQIAARC

SEQ ID NO: 55 - protein, 32 aa, artificial sequence
  synthetic CCR5 PNt CN (Cysteine at the N terminus)
CMDYQVSSPIYDINYYTSEPCQKINVKQIAAR

SEQ ID NO: 56 - protein, 5 aa, artificial sequence
  Ntb domain of synthetic CCR5 PNt
INVKQ

SEQ ID NO: 57 - protein, 19 aa, artificial sequence
  Nta domain of synthetic CCR5 PNt
MDYQVSSPIYDINYYTSEP

SEQ ID NO: 58 - protein, 12 aa, artificial sequence
  Ntb domain of synthetic CCR5 PNt
CQKINVKQIAAR

SEQ ID NO: 59 - protein, 7 aa, artificial sequence
  synthetic Ntb domain fragment
CQKINVK

SEQ ID NO: 60 - protein, 8 aa, artificial sequence
  synthetic Ntb domain fragment
CQKINVKQ

SEQ ID NO: 61 - protein, 5 aa, artificial sequence
  Modified HBcAg
GGKGG

SEQ ID NO: 62 - protein, 9 aa, artificial sequence
  misc_feature (1)..(1): Xaa can be Glu, pyro Glu or Gln
XGPWLEEEE

SEQ ID NO: 63 - protein, 7 aa, artificial sequence
  Linker sequence for modified gastrin 1-9 fragment
SSPPPPC

SEQ ID NO: 64 - protein, 24 aa, artificial sequence
  Synthetic sequence fused to C-terminus of AP205 VLP
GTAGGGSGFGFPEHLLVDFLQSLS

SEQ ID NO: 65 - protein, 5 aa, artificial sequence
  Synthetic N-terminal linker sequence
CGSGG

(19) United States
(12) Patent Application Publication
(10) Pub. No.: US 2010/0111995 A1
(43) Pub. Date: May 6, 2010

(54) Antigen conjugates and uses thereof

(75) Inventors: Martin F. Bachman, Seuzach (CH); Alain Tissot, Zurich (CH); Andrea Jegerlehner, Zurich (CH); Philippe Saudan, Pfungen (CH); Yu Zou, Birmensdorf (CH); Nicole Schmitz, Urdorf (CH); Adrian Huber, Zurich (CH); Stephen Martin, Dunedin (NZ); Heather Hinton, Zug (CH)

Correspondence Address: STERNE, KESSLER, GOLDSTEIN & FOX P.L.L.C., 1100 NEW YORK AVENUE, N.W., WASHINGTON, DC, 20005, US

(73) Assignee: Cytos Biotechnology AG, Zürich-Schlieren (CH)

(21) Appl. No.: 11/922,217

(22) PCT Filed: Jun. 14, 2006
(86) PCT No.: PCT/EP2006/063198

§ 371 Date: Jan. 13, 2010

Related U.S. Application Data
(60) Provisional application No. 60/690,094, filed on Jun. 14, 2005.

(51) Int. Cl. A61K 39/12; C07K 19/00; A61P 31/18; A61P 19/02; A61P 35/00

(52) U.S. Cl. 424/196.11; 530/350; 424/193.1

(57) ABSTRACT

The present invention is in the fields of medicine, public health, immunology, molecular biology and virology. The invention provides composition comprising a virus-like particle (VLP) linked to at least one antigen of the invention, wherein said antigen of the invention is CCR5 of the invention, gastrin of the invention, CXCR4 of the invention, CETP of the invention or C5a of the invention. The invention also provides a process for producing the composition. The compositions of this invention are useful in the production of vaccines, in particular, for the treatment of diseases in which the antigen of the invention mediates, or contributes to the condition, particularly for the treatment of AIDS, gastrointestinal cancers, coronary heart diseases or inflammatory diseases. Moreover, the compositions of the invention induce efficient immune responses, in particular antibody responses. Furthermore, the compositions of the invention are particularly useful to efficiently induce self-specific immune responses within the indicated context.

### BACKGROUND OF THE INVENTION

1. Field of the Invention

The present invention is in the fields of medicine, public health, immunology, molecular biology and virology. The invention provides composition comprising a virus-like particle (VLP) linked to at least one antigen of the invention, wherein said antigen of the invention is CCR5 of the invention, gastrin of the invention, CXCR4 of the invention, CETP of the invention or C5a of the invention.

The invention also provides a process for producing the composition. The compositions of this invention are useful in the production of vaccines, in particular, for the treatment of diseases in which the antigen of the invention mediates, or contributes to the condition, particularly for the treatment of AIDS, gastrointestinal cancers, coronary heart diseases or inflammatory diseases. Moreover, the compositions of the invention induce efficient immune responses, in particular antibody responses. Furthermore, the compositions of the invention are particularly useful to efficiently induce self-specific immune responses within the indicated context.

2. Related Art

HIV R5 strains use the cell surface molecules CD4 and CCR5 for attachment and entry into macrophages and CD4+ T cells. CCR5 is a 7-transmembrane receptor with an N-terminal sequence and three loops exposed to the extracellular space, which are called subsequently PNt, ECL-1, ECL-2, and ECL-3, respectively. The natural CCR5 ligands, RANTES, MIP-1α, MIP-1β and analogs thereof are able to block the virus-coreceptor interaction and further cause the internalization of CCR5 (Lederman et al., 2004, Science 306, p485). CCR5 specific auto-antibodies have been found in 12.5% women that were repeatedly exposed to HIV but remained uninfected (Lopalco et al., 2000, J. Immunology 164, 3426). These antibodies were shown to bind the first extracellular loop (ECL-1) of CCR5 and could inhibit R5-tropic HIV infection of peripheral blood mononuclear cells (PBMC). Alloimmunisation in women led to CCR5 specific antibodies that were capable of inhibiting R5-HIV infection in vitro (Wang et al., 2002, Clin. Exp. Immunol. 129, 493).

Monoclonal α-CCR5 antibodies are able to prevent HIV infection in vitro (Olson et al., 1999, J. Virol. 73, 4145; Wu and LaRosa et al., 1997, J. Exp. Med. 186, 1373). Antibody binding to a cyclic peptide corresponding to the small extracellular loop ECL-2A (Arg168-Cys178) suppressed infection by HIV-1 R5 (Misumi et al., 2001, J. Virol. 75, 11614). Antibodies produced by immunizing monkeys with linear CCR5 peptides (from the N-terminal, the ECL-1, or the ECL-2 sequence) have viral inhibitory effect in vitro (Lehner et al., 2001, J. Immunology 166, 7446). The N-terminal domain of CCR5 was displayed on papillomavirus like particles and immunized monkey (Chackerian et al., 2004, J. Virol. 78, 4037).

The chemokine receptor CXCR4, also known as LESTR or fusin, belongs to the family of seven-transmembrane domain G-protein coupled receptors (Federsppiel et. al. (1993), Genomics 16:707). CXCR4 is expressed on the cell surface of most leukocyte populations, including all B cells and monocytes, the majority of T-lymphocyte subsets, endothelial cells and epithelial cells (Murdoch, (2000) Immunol. Rev. 177:175). The only known ligand for CXCR4 is SDF-1 (Pelchen-Mattews, et. al. (1999) Immunol. Rev. 168:33).

CXCR4 was later identified as a co-receptor for HIV (Feng et al (1996) Science 272:872). Accordingly, HIV strains that necessity CXCR4 for entry into cells are categorized as X4 strain and this entry can be blocked by SDF-1 has been shown to block HIV-1 entry (Oberlin et al (1996), Nature 382:833; Bleul, et al (1996) Nature 382:829.

Several CXCR4 peptide antagonists have been identified and were shown to inhibit the entry and infection of X4 HIV-1 strains (Murakami et al (1997) J Exp Med 1863389; Arakaki et al (1999). J Virol 73:1719; Doranz et al (2001) AIDS Res Hum Retroviruses 17:475; Doranz, et al (1997) J Exp Med 186:1395; Schols, D. (2004), Curr Top Med Chem 4:883. In addition, the small chemical compound AMD3100 which is a potent and selective inhibitor of HIV-1 and HIV-2 replication has been shown to be specific for CXCR4 (De Clercq (2003) Nat Rev Drug Discov 2:581). Moreover anti-CXCR4 monoclonal antibodies targeting different extracellular domains of CXCR4 were shown to inhibit HIV-1 infection (Endres et al (1996) Cell 87:745; Brelot et al (1997), J Virol 71:4744; Misumi et al (2003), J Biol Chem 278:32335; Xiao et al (2000), Exp Mol Pathol 68:139).

Gastrin (G17) is a group of classical gut peptide hormones with much lower amount in the colon and pancreas (Koh, Regulatory Peptides. 93, 37-44 (2000)). Gastrin is processed from its precursor progastrin (G34). Both gastrin and progastrin exist in a C-terminal glycine-extended form and in a C-terminal phenylalanine amidated form. (Steel. IDrugs. 5, 689-695 (2002)).

Gastrin is well known for its ability to stimulate gastric acid secretion (Pharmacol Ther. 98, 109-127 (2003)). The related hormone cholecystokinin (CCK), which has the C-terminal tetrapeptide amide as gastrin, is synthesized in the duodenum and is responsible for pancreatic enzyme secretion. While amidated G17 binds to CCK-2 receptor, CCK binds to both CCK-1 receptor and CCK-2 receptors (Steel. IDrugs. 5, 689-695 (2002)). The receptor for the glycine-extended gastrin remains unclear. Recent data suggest that gastrin might promote the development of cancers of the gastrointestinal tract (Watson. Aliment Pharmacol Ther. 14, 1231-1247 (2000); Watson. Aliment Pharmacol Ther. 14, 1231-1247 (2000)).

Activation of the complement system induces a number of potent biological effects, many of which are mediated by the anaphylatoxin C5a. The fifth component of complement (C5) is cleaved by the C5 convertase into two fragments, C5a and C5b.

C5a, a 74-amino acid, 4-helix bundle glycoprotein (Fernandez and Hugh, J. Biol. Chem. 253, 6955-6964, 1978), is responsible for generating a number of diverse effects on cellular systems, especially neutrophils, endothelial cells and macrophages to induce local inflammation to combat infecting microorganisms (Ward P., Nat. Rev. Immunol. 4:133, 2004). However, by the same token, the excessive generation of C5a in sepsis leads to serious functional defects in neutrophils (Czermak et al., Nat. Med. 5:788, 1999; Huber-Lang et al., J. Immunol. 166:1193, 2001).

Elevated activation of C5a has been also implicated in a number of primary and/or chronic inflammatory diseases, such as rheumatoid arthritis (Jose P. Ann Rheum. Dis. 49:747, 1990), psoriasis (Takematsu H., Arch. Dermatol. 129:74, 1993), adult respiratory distress syndrome (Langlois P., Heart Lung 18:71, 1989), reperfusion injury (Homeister, J. Annu. Rev. Pharmacol. Toxicol. 34:17, 1994), lupus nephritis and bullous pemphigoid.

Antibodies, which bind to C5 and block the cleavage and thereby reduce the generation of C5a and C5b, have been suggested for use in treating conditions like, for example, glomerulonephritis (WO9529697), asthma (WO04022096), collagen-induced arthritis (Wang et al, Proc. Natl. Acad. Sci., 92:8955, 1995) and serum transferred arthritis (Ji et al, Immunity, 16:157, 2002). Antibodies, specifically binding to C5a, have been suggested to use in treating adult respiratory distress syndrome (ARDS) (WO8605692) and injurious intravascular complement activation (EP245993). The use of a monoclonal antibody, which is reactive to the extracellular loop of C5aR and thereby presumably reduces or inhibits the binding of C5a with C5aR, has also been proposed in treating immunopathological disorders (WO2003062278).

Cholesteryl-ester transfer protein (CETP) is a plasma glycoprotein which mediates the exchange of cholesterol ester (CE) and triglycerides (TG) between High density lipoprotein (HDL) particles and apo B rich particles such as very-low density liporprotein (VLDL) particles or low-density lipoprotein (LDL) particles. CETP also transfers phospholipids (PL). The human CETP cDNA encodes a protein of 476 amino acids.

HDL is considered anti-atherogenic, as an inverse correlation between HDL-cholesterol level and coronary heart disease (CHD) has been observed (Barter P. J. and Rye K.-A. (1996) Atherosclerosis 121: 1-12).

WO 96/39168 discloses a method for increasing HDL-c by stimulating an immune response that inhibits the activity of CETP. Immunization against CETP antigens has also been described in US2003/0026808. CETP polypeptides were fused to “MAPs”, and emulsified in Complete Freund's adjuvant (CFA) for immunization of rabbits. Fusion of a CETP peptide to Hepatitis B core antigen (HBcAg) has also been disclosed in US2003/0026808, but immunogenicity of the construct was not reported.

### SUMMARY OF THE INVENTION

We have, now, surprisingly found that the inventive compositions and vaccines, respectively, comprising at least one CCR5 extracellular domain or at least one CCR5 extracellular domain fragment, are capable of inducing immune responses, in particular antibody responses, leading to high antibody titer against CCR5. Moreover, we have surprisingly found that inventive compositions and vaccines, respectively, comprising at least one CCR5 extracellular domain or at least one CCR5 extracellular domain fragment, are capable of inducing immune responses, in particular antibody responses, with protective and/or therapeutic effect against the HIV infection. This indicates that the immune responses, in particular the antibodies generated by the inventive compositions and vaccines, respectively, are, thus, capable of specifically recognizing CCR5 in vivo, and interfering with its function as HIV co-receptor.

Thus, in the first aspect, the present invention provides a composition which comprises (a) a virus-like particle (VLP) with at least one first attachment site; and (b) at least one antigen with at least one second attachment site, wherein said at least one antigen is a CCR5 extracellular domain or a CCR5 extracellular domain fragment or any combination thereof, and wherein (a) and (b) are linked through said at least one first and said at least one second attachment site, preferably to form an ordered and repetitive antigen array. In one preferred embodiment of the invention, the virus-like particle suitable for use in the present invention comprises recombinant protein, preferably recombinant coat protein, mutants or fragments thereof, of a virus, preferably of a RNA bacteriophage.

In one preferred embodiment, the present invention provides a composition comprising: (a) a virus-like particle (VLP) of an RNA-bacteriophage with at least two first attachment sites; and (b) at least one CCR5 extracellular domain PNt with at least two second attachment sites; wherein said CCR5 extracellular domain PNt comprises, consists essentially of or consists of (i) a Nta domain or a Nta domain fragment, and (ii) a Ntb domain comprising amino acids 23 to 27 of SEQ ID NO:27 (SEQ ID NO:56) or Ntb domain fragment comprising amino acids 23 to 27 of SEQ ID NO:27, wherein the C-terminus of said Nta domain or said Nta domain fragment is fused, preferably directly, to said N-terminus of said Ntb domain or said Ntb domain fragment, and wherein the first or the second of said at least two second attachment sites comprises or is a sulthydryl group, preferably a sulfhydryl group of a cysteine residue, and wherein the first of said at least two second attachment sites is located upstream of the N-terminus of said amino acids 23 to 27 of SEQ ID NO:27; and wherein the second of said at least two second attachment sites is located downstream of the C terminus of said amino acids 23 to 27 of SEQ ID NO:27, preferably downstream of the C terminus of said CCR5 extracellular domain PNt; and wherein said VLP of said RNA-bacteriophage and said CCR5 extracellular domain PNt are linked by at least one non-peptide covalent bond.

In another aspect, the present invention provides a method of preventing and/or treating HIV infection, wherein the method comprises administering the inventive composition or the inventive vaccine composition, respectively, to a human, wherein the antigen of the invention is a CCR5 of the invention.

We have, now, surprisingly found that the inventive compositions and vaccines, respectively, comprising at least one CXCR4 extracellular domain or at least one CXCR4 extracellular domain fragment, are capable of inducing strong immune responses, in particular strong antibody responses, leading to high antibody titer against CXCR4.

Thus, in the first aspect, the present invention provides a composition which comprises (a) a virus-like particle (VLP) with at least one first attachment site; and (b) at least one antigen with at least one second attachment site, wherein said at least one antigen is a CXCR4 extracellular domain or a CXCR4 extracellular domain fragment or any combination thereof, and wherein (a) and (b) are linked through said at least one first and said at least one second attachment site, preferably to form an ordered and repetitive antigen array. In one preferred embodiment of the invention, the virus-like particle suitable for use in the present invention comprises recombinant protein, preferably recombinant coat protein, mutants or fragments thereof, of a virus, preferably of a RNA bacteriophage.

We have, now, surprisingly found that the inventive compositions and vaccines, respectively, comprising at least one CETP protein or at least one CETP fragment, are capable of inducing strong immune responses, in particular strong antibody responses, leading to high antibody titer against CETP.

Thus, in the first aspect, the present invention provides a composition which comprises (a) a virus-like particle (VLP) with at least one first attachment site; and (b) at least one antigen with at least one second attachment site, wherein said at least one antigen is a CETP protein or a CETP fragment and wherein (a) and (b) are linked through said at least one first and said at least one second attachment site, preferably to form an ordered and repetitive antigen array. In one preferred embodiment of the invention, the virus-like particle suitable for use in the present invention comprises recombinant protein, preferably recombinant coat protein, mutants or fragments thereof, of a virus, preferably of a RNA bacteriophage.

We have, now, surprisingly found that the inventive compositions and vaccines, respectively, comprising at least one C5a protein or at least one C5a fragment, are capable of inducing strong immune responses, in particular strong antibody responses, leading to high antibody titer against C5a. Moreover, we have surprisingly found that inventive compositions and vaccines, respectively, comprising at least one C5a protein or at least one C5a fragment, are capable of inducing strong immune responses, in particular strong antibody responses, with protective and/or therapeutic effect against primary and/or chronic inflammatory diseases, in which C5a plays an important role, such as arthritis. This indicates that the immune responses, in particular the antibodies generated by the inventive compositions and vaccines, respectively, are, thus, capable of specifically recognizing C5a in vivo, and interfering with its function.

Thus, in the first aspect, the present invention provides a composition which comprises (a) a virus-like particle (VLP) with at least one first attachment site; and (b) at least one antigen with at least one second attachment site, wherein said at least one antigen is a C5a protein or a C5a fragment and wherein (a) and (b) are linked through said at least one first and said at least one second attachment site, preferably to form an ordered and repetitive antigen array. In one preferred embodiment of the invention, the virus-like particle suitable for use in the present invention comprises recombinant protein, preferably recombinant coat protein, mutants or fragments thereof, of a virus, preferably of a RNA bacteriophage.

The present invention is advantageous over prior art employing monoclonal antibodies against C5a for treating diseases. Shortcomings of monoclonal antibody therapy include the need for repeated injections of large amounts of antibody (Kaplan, Curr Opin Invest. Drugs. 2002; 3:1017-23). High doses of antibodies can lead to side-effects such as infusion disease. Anti-antibodies can also be generated in patients in an allotypic response, even if human or humanized antibodies are used, leading to a decreased therapeutic effect or potentially also causing side-effects. Moreover, the expense associated with the high production cost of humanized monoclonal antibody and with the need for frequent hospital visit renders this antibody treatment unavailable to many patients in need.

In one aspect, the present invention provides a method of preventing and/or treating primary and/or chronic inflammatory diseases, wherein the method comprises administering the inventive composition or the invention vaccine composition, respectively, to an animal or a human, wherein the antigen of the invention is a C5a of the invention. Primary and/or chronic inflammatory diseases, in which C5a mediates or contributes to the condition, include but are not limited to rheumatoid arthritis, systemic lupus erythematosus, asthma and bullous pemphigoid.

In one aspect, the present invention provides a composition which comprises (a) a virus-like particle (VLP) with at least one first attachment site; and (b) at least one antigen with at least one second attachment site, wherein said at least one antigen is Bradykinin of the invention, and wherein (a) and (b) are linked through said at least one first and said at least one second attachment site, preferably to form an ordered and repetitive antigen array. In one preferred embodiment of the invention, the virus-like particle suitable for use in the present invention comprises recombinant protein, preferably recombinant coat protein, mutants or fragments thereof, of a virus, preferably of a RNA bacteriophage.

In one aspect, the present invention provides a composition which comprises (a) a virus-like particle (VLP) with at least one first attachment site; and (b) at least one antigen with at least one second attachment site, wherein said at least one antigen is des-Arg-Bradykinin of the invention, and wherein (a) and (b) are linked through said at least one first and said at least one second attachment site, preferably to form an ordered and repetitive antigen array. In one preferred embodiment of the invention, the virus-like particle suitable for use in the present invention comprises recombinant protein, preferably recombinant coat protein, mutants or fragments thereof, of a virus, preferably of a RNA bacteriophage.

We have, now, surprisingly found that the inventive compositions and vaccines, respectively, comprising at least one gastrin G17, at least one fragment of gastrin G17, progastrin G34, or at least one fragment of progastrin G34, are capable of inducing strong immune responses, in particular strong antibody responses, leading to high antibody titer against gastrin or progastrin.

Thus, in the first aspect, the present invention provides a composition which comprises (a) a virus-like particle (VLP) with at least one first attachment site; and (b) at least one antigen with at least one second attachment site, wherein said at least one antigen is a gastrin G17, a fragment of gastrin G17, a progastrin G34, or a fragment of progastrin G34 and wherein (a) and (b) are linked through said at least one first and said at least one second attachment site, preferably to form an ordered and repetitive antigen array. In one preferred embodiment of the invention, the virus-like particle suitable for use in the present invention comprises recombinant protein, preferably recombinant coat protein, mutants or fragments thereof, of a virus, preferably of a RNA bacteriophage.

In another aspect, the present invention provides a vaccine composition, wherein said vaccine composition comprises at least one antigen of the invention. Furthermore, the present invention provides a method to administering the vaccine composition to a human or an animal, preferably a mammal. The inventive vaccine composition is capable of inducing strong immune response, in particular antibody response, without the presence of at least one adjuvant. Thus, in one preferred embodiment, the vaccine composition is devoid of an adjuvant. The avoidance of using adjuvant may reduce a possible occurrence of unwanted inflammatory T cell responses.

In a further aspect, the present invention provides a pharmaceutical composition comprising the inventive composition and an acceptable pharmaceutical carrier.

In again a further aspect, the present invention provides for a method of producing the composition of the invention comprising (a) providing a VLP with at least one first attachment site; (b) providing at least one antigen of the invention with at least one second attachment site; and (c) combining said VLP and said at least one antigen of the invention to produce said composition, wherein said at least one antigen and said VLP are linked through said at least one first and said at least one second attachment sites.

### BRIEF DESCRIPTION OF THE FIGURES

FIG. 1A shows the results of ELISA of plates coated with either nG17amide or CCK8 and incubated with serially diluted mouse sera (14 days after immunization). FIG. 1B shows the results of inhibition ELISA, in which the sera was preincubated with serially diluted nG17amide or CCK8 before added to the coated plates.

FIG. 2 shows the average clinical score sum across all limbs after the final collagen/CFA injection (FIG. 2A) or after final anti-collagen-monoclonal antibody-cocktail injection (FIG. 2B) of mice immunized with either Qβ-mC5acys or Qβ VLP. The x-axis represents the days after collagen injection and the y-axis represents the average sum of clinical score of all legs.

FIG. 3 shows percentage of mice immunized with either Qβ-mC5acys or Qβ VLP with proteinuria readings of greater than 300 μg/ml.

### DETAILED DESCRIPTION OF THE INVENTION

Unless defined otherwise, all technical and scientific terms used herein have the same meanings as commonly understood by one of ordinary skill in the art to which this invention belongs.

Antigen: As used herein, the term “antigen” refers to a molecule capable of being bound by an antibody or a T cell receptor (TCR) if presented by MHC molecules. The term “antigen”, as used herein, also encompasses 1-cell epitopes. An antigen is additionally capable of being recognized by the immune system and/or being capable of inducing a humoral immune response and/or cellular immune response leading to the activation of B- and/or 1-lymphocytes. This may, however, require that, at least in certain cases, the antigen contains or is linked to a Th cell epitope and is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens.

Antigenic site: The term “antigenic site” and the term “antigenic epitope”, which are used herein interchangeably, refer to continuous or discontinuous portions of a polypeptide, which can be bound immunospecifically by an antibody or by a T-cell receptor within the context of an MHC molecule. Immunospecific binding excludes non-specific binding but does not necessarily exclude cross-reactivity. Antigenic site typically comprise 5-10 amino acids in a spatial conformation which is unique to the antigenic site.

Antigen of the invention: the term “antigen of the invention”, as used herein, refers to an antigen selected from the group consisting of: a) CCR5 of the invention; b) CXCR4 of the invention; c) CETP of the invention; d) C5a of the invention; c) gastrin of the invention, f) Bradykinin of the invention; and g) des-Arg-Bradykinin of the invention.

CCR5 of the invention: The term “CCR5 of the invention” as used herein, refers to at least one CCR5 extracellular domain, at least one CCR5 extracellular domain fragment as defined herein or any combination thereof.

CCR5 extracellular domain: The term “CCR5 extracellular domain” as used herein should encompass any polypeptide comprising, consisting essentially of, or alternatively or preferably consisting of, any one of the four extracellular domains of human CCR5 of SEQ ID NO:24 or the corresponding orthologs from any other animals, preferably mammals. Moreover, the term “CCR5 extracellular domain” as used herein should also encompass any polypeptide comprising, consisting essentially of, or alternatively or preferably consisting of, any natural or genetically engineered variant having more than 70%, preferably more than 80%, even more preferably more than 90%, again more preferably more than 95%, and most preferably more than 97% amino acid sequence identity with the CCR5 extracellular domain as defined above. The term “CCR5 extracellular domain” as used herein should furthermore encompass post-translational modifications including but not limited to glycosylations, acetylations, phosphorylations of the CCR5 extracellular domain as defined above. Preferably the CCR5 extracellular domain, as defined herein, consists of at most 200, even more preferably at most 100 amino acids in length. Typically and preferably, CCR5 extracellular domain is capable of inducing in vivo the production of antibody specifically binding to CCR5.

CCR5 extracellular domain fragment: The term “CCR5 extracellular domain fragment” as used herein should encompass any polypeptide comprising, consisting essentially of, or alternatively or preferably consisting of, at least 4, 5, preferably at least 6, 7, 8, 9, 10, 11, 12, 17, 18, 19, 20, 25, or 30 contiguous amino acids of a CCR5 extracellular domain as defined herein as well as any polypeptide having more than 65%, preferably more than 80%, more preferably more than 90% and even more preferably more than 95% amino acid sequence identity thereto. Preferably, the term “CCR5 extracellular domain fragment” as used herein should encompass any polypeptide comprising, consisting essentially of, or alternatively or preferably consisting of, at least 6 contiguous amino acids of a CCR5 extracellular domain as defined herein as well as any polypeptide having more than 65%, preferably more than 80%, preferably more than 90% and even more preferably more than 95% amino acid sequence identity thereto. Preferably the CCR5 extracellular domain fragment, as defined herein, consists of at most 50, even more preferably at most 30 amino acids in length. Typically and preferably, a CCR5 extracellular domain fragment is capable of inducing the production of antibody in vivo, which specifically binds to CCR5.

Combination of CCR5 extracellular domain and/or CCR5 extracellular domain fragment: the term “combination of CCR5 extracellular domain and/or CCR5 extracellular domain fragment” should encompass any entity comprising or alternatively consisting of any combination of CCR5 extracellular domain and/or CCR5 extracellular domain fragment as defined above. Preferably CCR5 extracellular domain and/or CCR5 extracellular domain fragment are combined by fusion into one polypeptide. Thus, the term “combination of CCR5 extracellular domain and/or CCR5 extracellular domain fragment” further comprises additional amino acids as spacer, wherein said spacer is usually not longer than 10, preferably not longer than 6 amino acids and wherein said spacer is in between two CCR5 extracellular domains and/or CCR5 extracellular domain fragments.

CXCR4 of the invention: The term “CXCR4 of the invention” as used herein, refers to at least one CXCR4 extracellular domain, at least one CXCR4 extracellular domain fragment as defined herein or any combination thereof.

CXCR4 extracellular domain: The term “CXCR4 extracellular domain” as used herein should encompass any polypeptide comprising, consisting essentially of, or alternatively or preferably consisting of, any one of the four extracellular domains of human CXCR4 of SEQ ID NO:28 or the corresponding orthologs from any other animals, preferably mammals. Moreover, the term “CXCR4 extracellular domain” as used herein should also encompass any polypeptide comprising, consisting essentially of, or alternatively or preferably consisting of, any natural or genetically engineered variant having more than 70%, preferably more than 80%, even more preferably more than 90%, again more preferably more than 95%, and most preferably more than 97% amino acid sequence identity with the CXCR4 extracellular domain as defined above. The term “CXCR4 extracellular domain” as used herein should furthermore encompass post-translational modifications including but not limited to glycosylations, acetylations, phosphorylations of the CXCR4 extracellular domain as defined above. Preferably the CXCR4 extracellular domain, as defined herein, consists of at most 200, even more preferably of at most 100 amino acids in length. Typically and preferably, CXCR4 extracellular domain is capable of inducing in vivo the production of antibody specifically binding to CXCR4.

CXCR4 extracellular domain fragment: The term “CXCR4 extracellular domain fragment” as used herein should encompass any polypeptide comprising, consisting essentially of, or alternatively or preferably consisting of, at least 4, 5, preferably at least 6, 7, 8, 9, 10, 11, 12, 17, 18, 19, 20, 25, or 30 contiguous amino acids of a CXCR4 extracellular domain as defined herein as well as any polypeptide having more than 65%, preferably more than 80%, more preferably more than 90% and even more preferably more than 95% amino acid sequence identity thereto. Preferably, the term “CXCR4 extracellular domain” as used herein should encompass any polypeptide comprising, consisting essentially of, or alternatively or preferably consisting of, at least 6 contiguous amino acids of a CXCR4 extracellular domain as defined herein as well as any polypeptide having more than 65%, preferably more than 80%, preferably more than 90% and even more preferably more than 95% amino acid sequence identity thereto. Preferably the CXCR4 extracellular domain fragment, as defined herein, consists of at most 50, even more preferably of at most 30 amino acids in length. Typically and preferably, a CXCR4 extracellular domain fragment is capable of inducing the production of antibody in vivo, which specifically binds to CXCR4.

Combination of CXCR4 extracellular domain and/or CXCR4 extracellular domain fragment: the term “Combination of CXCR4 extracellular domain and/or CXCR4 extracellular domain fragment” encompasses any entity comprising or alternatively consisting of any combination of CXCR4 extracellular domain and/or CXCR4 extracellular domain fragment as defined above. Preferably CXCR4 extracellular domain and/or CXCR4 extracellular domain fragment are combined by fusion into one polypeptide. Thus, the term “combination of CXCR4 extracellular domain and/or CXCR4 extracellular domain fragment” further comprises additional amino acids as spacer, wherein said spacer is usually not longer than 10, preferably not longer than 6 amino acids and wherein said spacer is in between two CXCR4 extracellular domains and/or CXCR4 extracellular domain fragments.

C5a of the invention: The term “C5a of the invention” as used herein, refers to at least one C5a protein or at least one C5a fragment as defined herein or any combination thereof.

C5a protein: The term “C5a protein” as used herein should encompass any polypeptide comprising, consisting essentially of, or alternatively or preferably consisting of, the human C5a of SEQ ID NO:45 or the corresponding orthologs from any other animals, preferably mammals. Moreover, the term “C5a protein” as used herein should also encompass any polypeptide comprising, consisting essentially of, or alternatively or preferably consisting of, any natural or genetically engineered variant having more than 70%, preferably more than 80%, even more preferably more than 90%, again more preferably more than 95%, and most preferably more than 97% amino acid sequence identity with the human C5a of SEQ ID NO:45 or the corresponding orthologs from any other animals. The term “C5a protein” as used herein should furthermore encompass post-translational modifications including but not limited to glycosylations, acetylations, phosphorylations of the C5a protein as defined above. Preferably the C5a protein, as defined herein, consists of at most 200, even more preferably of at most 100 amino acids in length. Typically and preferably, C5a protein is capable of inducing in vivo the production of antibody specifically binding to C5a.

C5a fragment: The term “C5a fragment” as used herein should encompass any polypeptide comprising, consisting essentially of, or alternatively or preferably consisting of, at least 4, 5, preferably at least 6, 7, 8, 9, 10, 11, 12, 17, 18, 19, 20, 25 or 30 contiguous amino acids of a C5a protein as defined herein as well as any polypeptide having more than 65%, preferably more than 80%, more preferably more than 90% and even more preferably more than 95% amino acid sequence identity thereto. Preferably, the term “C5a fragment” as used herein should encompass any polypeptide comprising, consisting essentially of, or alternatively or preferably consisting of, at least 6 contiguous amino acids of a C5a protein as defined herein as well as any polypeptide having more than 65%, preferably more than 80%, preferably more than 90% and even more preferably more than 95% amino acid sequence identity thereto. Preferably the C5a fragment, as defined herein, consists of at most 50, even more preferably of at most 30 amino acids in length. Typically and preferably, a C5a fragment is capable of inducing the production of antibody in vivo, which specifically binds to C5a.

CETP of the invention: The term “CETP of the invention” as used herein, refers to at least one CETP protein or at least one CETP fragment as defined herein or any combination thereof.

CETP protein: The term “CETP protein” as used herein should encompass any polypeptide comprising, consisting essentially of, or alternatively or preferably consisting of, the human CETP of SEQ ID NO:31 or the corresponding orthologs from any other animals, preferably mammals. Moreover, the term “CETP protein” as used herein should also encompass any polypeptide comprising, consisting essentially of, or alternatively or preferably consisting of, any natural or genetically engineered variant having more than 70%, preferably more than 80%, even more preferably more than 90%, again more preferably more than 95%, and most preferably more than 97% amino acid sequence identity with the human CETP of SEQ ID NO:31 or the corresponding orthologs from any other animals. The term “CETP protein” as used herein should furthermore encompass post-translational modifications including but not limited to glycosylations, acetylations, phosphorylations of the CETP protein as defined above. Preferably the CETP protein, as defined herein, consists of at most 500 amino acids in length. Typically and preferably, CETP protein is capable of inducing in vivo the production of antibody specifically binding to CETP.

CETP fragment: The term “CETP fragment” as used herein should encompass any polypeptide comprising, consisting essentially of, or alternatively or preferably consisting of, at least 4, 5, preferably at least 6, 7, 8, 9, 10, 11, 12, 17, 18, 19, 20, 25 or 30 contiguous amino acids of a CETP protein as defined herein as well as any polypeptide having more than 65%, preferably more than 80%, more preferably more than 90% and even more preferably more than 95% amino acid sequence identity thereto. Preferably, the term “CETP fragment” as used herein should encompass any polypeptide comprising, consisting essentially of, or alternatively or preferably consisting of, at least 6 contiguous amino acids of a CETP protein as defined herein as well as any polypeptide having more than 80%, preferably more than 90% and even more preferably more than 95% amino acid sequence identity thereto. Preferably the CETP fragment, as defined herein, consists of at most 50, even more preferably of at most 30 amino acids in length. Typically and preferably, a CETP fragment is capable of inducing the production of antibody in vivo, which specifically binds to CETP.

Gastrin of the invention: The term “gastrin of the invention” as used herein, refers to at least one gastrin G17, at least one fragment of gastrin G17, at least one progastrin G34 or at least one fragment of progastrin G34 as defined herein or any combination thereof.

Gastrin G17: The term “gastrin G17” should encompass any polypeptide comprising, consisting essentially of, or alternatively consisting of the human gastrin 1-17 as of SEQ ID NO:34, SEQ ID NO: 36, gastrin 1-17 of SEQ ID NO:34 with the C-terminal phenylalanine amidated or the corresponding orthologs from any other animals, preferably mammals. The term “gastrin G17” should further encompass any polypeptide comprising, consisting essentially of, or alternatively consisting of the human gastrin 1-17 as of SEQ ID NO:34, SEQ ID NO: 36, gastrin 1-17 of SEQ ID NO:34 with the C-terminal phenylalanine amidated or the corresponding orthologs from any other animals, in which at most three, preferably at most two, more preferably one amino acid has been deleted, added or substituted. Preferably the substitution is conservative amino acid substitution. The length of gastrin G17 is preferably not longer than 50, more preferably not longer than 30 amino acids. Typically and preferably, a gastrin G17 is capable of inducing the production of antibody in vivo, which specifically binds to gastrin.

Fragment of gastrin G17: the term “fragment of grstrin G17” as used herein, should encompasses any polypeptide comprising, consisting essentially of, or alternatively consisting of at least 4, 5, preferably at least 6, 7, 8, 9, or 10 contiguous amino acids of gastrin G17. The term “fragment of gastrin G17” should further encompass any polypeptide comprising, consisting essentially of, or alternatively consisting of fragment of grstrin G17 as defined above, in which at least one amino acid, preferably at most 3, even more preferably at most 2, even more preferably one amino acid has been deleted, added or substitute by another amino acid. Preferably the substitution is conservative amino acid substitution. The length of a fragment of gastrin G17 is preferably not longer than 30, more preferably not longer than 20 amino acids. Typically and preferably, a fragment of gastrin G17 is capable of inducing the production of antibody in vivo, which specifically binds to gastrin.

Progastrin G34: The term “progastrin G34” encompasses any polypeptide comprising, consisting essentially of, or alternatively consisting of the human gastrin 1-34 as of SEQ ID NO:35, SEQ ID NO: 37, gastrin 1-34 with the C-terminal phenylalanine amidated or the corresponding orthologs from any other animals, preferably mammals. The term “progastrin G34” should further encompass any polypeptide comprising, consisting essentially of, or alternatively consisting of the human gastrin 1-34 as of SEQ ID NO:35, SEQ ID NO: 37, gastrin 1-34 with the C-terminal phenylalanine amidated or the corresponding orthologs from any other animals, in which at most five, preferably at most four, more preferably at most three, preferably at most two, more preferably one amino acid has been deleted, added or substituted. Preferably the substitution is conservative substitution. The length of progastrin G34 is preferably not longer than 60, more preferably not longer than 40 amino acids. Typically and preferably, a progastrin G34 is capable of inducing the production of antibody in vivo, which specifically binds to progastrin.

Fragment of progastrin G34: the term “fragment of progastrin G34” as used herein, should encompasses any polypeptide comprising, consisting essentially of, or alternatively consisting of at least 6, 7, 8, 9, 10, 11, 12, 13 or 14 amino acid of progastrin G34. The term “fragment of progastrin G34” should further encompass any polypeptide comprising, consisting essentially of, or alternatively consisting of fragment of progastrin G34 as defined above, in which at least one amino acid, preferably at most 3, even more preferably at most 2, even more preferably one amino acid has been deleted, added or substitute by another amino acid. Preferably the substitution is conservative amino acid substitution. The length of a fragment of progastrin G34 is not longer than 40, more preferably not longer than 20 amino acids. Typically and preferably, a fragment of gastrin G34 is capable of inducing the production of antibody in vivo, which specifically binds to progastrin.

Bradykinin of the invention: the term “Bradykinin of the invention” as used herein, should encompass any polypeptide comprising, consisting essentially of, or alternatively consisting of the human Bradykinin as SEQ ID NO:22 or the corresponding orthologs from any other animals, preferably mammals. The term “Bradykinin of the invention” as used herein, should further encompass any polypeptide comprising, consisting essentially of, or alternatively consisting of the human Bradykinin as SEQ ID NO:22 or the corresponding orthologs from any other animals, in which at most two, preferably one amino acid has been deleted, added or substituted by another amino acid. Preferably the substitution is conservative amino acid substitution. The length of Bradykinin of the invention is preferably not longer than 30, more preferably not longer than 20 amino acids. Typically and preferably, a Bradykinin of the invention is capable of inducing the production of antibody in vivo, which specifically binds to Bradykinin.

Des-Arg-Bradykinin of the invention: the term “des-Arg-Bradykinin of the invention” as used herein, encompasses any polypeptide comprising, consisting essentially of, or alternatively consisting of the human des-Arg-Bradykinin as SEQ ID NO:23 or the corresponding orthologs from any other animals, preferably mammals. The term “des-Arg-Bradykinin of the invention” as used herein, further encompasses any polypeptide comprising, consisting essentially of, or alternatively consisting of the human des-Arg-Bradykinin as SEQ ID NO:23 or the corresponding orthologs from any other animal, in which at most two, preferably one amino acid has been deleted, added or substituted by another amino acid. Preferably the substitution is conservative amino acid substitution. The length of des-Arg-Bradykinin of the invention is preferably not longer than 30, more preferably not longer than 20 amino acids. Typically and preferably, a Bradykinin of the invention is capable of inducing the production of antibody in vivo, which specifically binds to des-Arg-Bradykinin.

Associated: The term “associated” (or its noun association) as used herein refers to all possible ways, preferably chemical interactions, by which two molecules are joined together. Chemical interactions include covalent and non-covalent interactions. Typical examples for non-covalent interactions are ionic interactions, hydrophobic interactions or hydrogen bonds, whereas covalent interactions are based, by way of example, on covalent bonds such as ester, ether, phosphoester, amide, peptide, carbon-phosphorus bonds, carbon-sulfur bonds such as thioether, or imide bonds.

Attachment Site, First: As used herein, the phrase “first attachment site” refers to an element which is naturally occurring with the VLP or which is artificially added to the VLP, and to which the second attachment site may be linked. The first attachment site may be a protein, a polypeptide, an amino acid, a peptide, a sugar, a polynucleotide, a natural or synthetic polymer, a secondary metabolite or compound (biotin, fluorescein, retinol, digoxigenin, metal ions, phenylmethylsulfonylfluoride), or a chemically reactive group such as an amino group, a carboxyl group, a sulfhydryl group, a hydroxyl group, a guanidinyl group, histidinyl group, or a combination thereof. A preferred embodiment of a chemically reactive group being the first attachment site is the amino group of an amino acid such as lysine. The first attachment site is located, typically on the surface, and preferably on the outer surface of the VLP. Multiple first attachment sites are present on the surface, preferably on the outer surface of virus-like particle, typically in a repetitive configuration. In a preferred embodiment the first attachment site is associated with the VLP, through at least one covalent bond, preferably through at least one peptide bond. In a further preferred embodiment the first attachment site is naturally occurring with the VLP. Alternatively, in another preferred embodiment the first attachment site is artificially added to the VLP.

Attachment Site, Second: As used herein, the phrase “second attachment site” refers to an element which is naturally occurring with or which is artificially added to the antigen of the invention and to which the first attachment site may be linked. The second attachment site of antigen of the invention may be a protein, a polypeptide, a peptide, an amino acid, a sugar, a polynucleotide, a natural or synthetic polymer, a secondary metabolite or compound (biotin, fluorescein, retinol, digoxigenin, metal ions, phenylmethylsulfonylfluoride), or a chemically reactive group such as an amino group, a carboxyl group, a sulthydryl group, a hydroxyl group, a guanidinyl group, histidinyl group, or a combination thereof. A preferred embodiment of a chemically reactive group being the second attachment site is the sulfhydryl group, preferably of an amino acid cysteine. The terms “antigen of the invention with at least one second attachment site”, as used herein, refers, to a construct comprising the antigen of the invention and at least one second attachment site. In one preferred embodiment, the second attachment site is naturally occurring within the antigen of the invention. In another preferred embodiment, the second attachment site is artificially added to the antigen of the invention. In one preferred embodiment the second attachment site is associated with the antigen of the invention through at least one covalent bond, preferably through at least one peptide bond. In one preferred embodiment, antigen of the invention with at least one second attachment site further comprises a linker, preferably said linker comprises at least one second attachment site, preferably said linker is fused to the antigen of the invention by a peptide bond.

Coat protein: The term “coat protein” and the interchangeably used term “capsid protein” within this application, refers to a viral protein, which is capable of being incorporated into a virus capsid or a VLP. Typically and preferably the term “coat protein” refers to the coat protein encoded by the genome of a virus, preferably an RNA bacteriophage or by the genome of a variant of a virus, preferably of an RNA bacteriophage. More preferably and by way of example, the term “coat protein of AP205” refers to SEQ ID NO:14 or the amino acid sequence, wherein the first methionine is cleaved from SEQ ID NO:14. More preferably and by way of example, the term “coat protein of Qβ” refers to SEQ ID NO:1 (“Qβ CP”) and SEQ ID NO:2 (A1), with or without the methione at the N-terminus. The capsid of bacteriophage Qβ is composed mainly of the Qβ CP, with a minor content of the A1 protein.

Linked: The term “linked” (or its noun: linkage) as used herein, refers to all possible ways, preferably chemical interactions, by which the at least one first attachment site and the at least one second attachment site are joined together. Chemical interactions include covalent and non-covalent interactions. Typical examples for non-covalent interactions are ionic interactions, hydrophobic interactions or hydrogen bonds, whereas covalent interactions are based, by way of example, on covalent bonds such as ester, ether, phosphoester, amide, peptide, carbon-phosphorus bonds, carbon-sulfur bonds such as thioether, or imide bonds. In certain preferred embodiments the first attachment site and the second attachment site are linked through at least one covalent bond, preferably through at least one non-peptide bond, and even more preferably through exclusively non-peptide bond(s). The term “linked” as used herein, however, shall not only encompass a direct linkage of the at least one first attachment site and the at least one second attachment site but also, alternatively and preferably, an indirect linkage of the at least one first attachment site and the at least one second attachment site through intermediate molecule(s), and hereby typically and preferably by using at least one, preferably one, heterobifunctional cross-linker.

Linker: A “linker”, as used herein, either associates the second attachment site with antigen of the invention or already comprises, essentially consists of, or consists of the second attachment site. Preferably, a “linker”, as used herein, already comprises the second attachment site, typically and preferably—but not necessarily—as one amino acid residue, preferably as a cysteine residue. A “linker” as used herein is also termed “amino acid linker”, in particular when a linker according to the invention contains at least one amino acid residue. Thus, the terms “linker” and “amino acid linker” are interchangeably used herein. However, this does not imply that such a linker consists exclusively of amino acid residues, even if a linker consisting of amino acid residues is a preferred embodiment of the present invention. The amino acid residues of the linker are, preferably, composed of naturally occurring amino acids or unnatural amino acids known in the art, all-L or all-D or mixtures thereof. Further preferred embodiments of a linker in accordance with this invention are molecules comprising a sulfhydryl group or a cysteine residue and such molecules are, therefore, also encompassed within this invention. Further linkers useful for the present invention are molecules comprising a C1-C6 alkyl-, a cycloalkyl such as a cyclopentyl or cyclohexyl, a cycloalkenyl, aryl or heteroaryl moiety. Moreover, linkers comprising preferably a C1-C6 alkyl-, cycloalkyl- (C5, C6), aryl- or heteroaryl-moiety and additional amino acid(s) can also be used as linkers for the present invention and shall be encompassed within the scope of the invention. Association of the linker with the antigen of the invention is preferably by way of at least one covalent bond, more preferably by way of at least one peptide bond. In case of a second attachment site not naturally occurring with the antigen of the invention, the linker is associated to the at least one second attachment site, for example, a cysteine, preferably, by way of at least one covalent bond, more preferably by way of at least one peptide bond.

Ordered and repetitive antigen array: As used herein, the term “ordered and repetitive antigen array” generally refers to a repeating pattern of antigen or, characterized by a typically and preferably high order of uniformity in spacial arrangement of the antigens with respect to virus-like particle, respectively. In one embodiment of the invention, the repeating pattern may be a geometric pattern. Certain embodiments of the invention, such as VLP of RNA phages, are typical and preferred examples of suitable ordered and repetitive antigen arrays which, moreover, possess strictly repetitive paracrystalline orders of antigens, preferably with spacings of 1 to 30 nanometers, preferably 2 to 15 nanometers, even more preferably 2 to 10 nanometers, even again more preferably 2 to 8 nanometers, and further more preferably 1.6 to 7 nanometers.

Packaged: The term “packaged” as used herein refers to the state of a polyanionic macromolecule in relation to the VLP. The term “packaged” as used herein includes binding that may be covalent, e.g., by chemically coupling, or non-covalent, e.g., ionic interactions, hydrophobic interactions, hydrogen bonds, etc. The term also includes the enclosement, or partial enclosement, of a polyanionic macromolecule. Thus, the polyanionic macromolecule can be enclosed by the VLP without the existence of an actual binding, in particular of a covalent binding. In preferred embodiments, the at least one polyanionic macromolecule is packaged inside the VLP, most preferably in a non-covalent manner.

Polypeptide: The term “polypeptide” as used herein refers to a molecule composed of monomers (amino acids) linearly linked by amide bonds (also known as peptide bonds). It indicates a molecular chain of amino acids and does not refer to a specific length of the product. Thus, peptides, dipeptides, tripeptides, oligopeptides and proteins are included within the definition of polypeptide. Post-translational modifications of the polypeptide, for example, glycosylations, acetylations, phosphorylations, and the like are also encompassed.

Recombinant VLP: The term “recombinant VLP”, as used herein, refers to a VLP that is obtained by a process which comprises at least one step of recombinant DNA technology. The term “VLP recombinantly produced”, as used herein, refers to a VLP that is obtained by a process which comprises at least one step of recombinant DNA technology. Thus, the terms “recombinant VLP” and “VLP recombinantly produced” are interchangeably used herein and should have the identical meaning.

Virus particle: The term “virus particle” as used herein refers to the morphological form of a virus. In some virus types it comprises a genome surrounded by a protein capsid; others have additional structures (e.g., envelopes, tails, etc.).

Virus-like particle (VLP), as used herein, refers to a non-replicative or non-infectious, preferably a non-replicative and non-infectious virus particle, or refers to a non-replicative or non-infectious, preferably a non-replicative and non-infectious structure resembling a virus particle, preferably a capsid of a virus. The term “non-replicative”, as used herein, refers to being incapable of replicating the genome comprised by the VLP. The term “non-infectious”, as used herein, refers to being incapable of entering the host cell. Preferably a virus-like particle in accordance with the invention is non-replicative and/or non-infectious since it lacks all or part of the viral genome or genome function. In one embodiment, a virus-like particle is a virus particle, in which the viral genome has been physically or chemically inactivated. Typically and more preferably a virus-like particle lacks all or part of the replicative and infectious components of the viral genome. A virus-like particle in accordance with the invention may contain nucleic acid distinct from their genome. A typical and preferred embodiment of a virus-like particle in accordance with the present invention is a viral capsid such as the viral capsid of the corresponding virus, bacteriophage, preferably RNA-phage. The terms “viral capsid” or “capsid”, refer to a macromolecular assembly composed of viral protein subunits. Typically, there are 60, 120, 180, 240, 300, 360 and more than 360 viral protein subunits. Typically and preferably, the interactions of these subunits lead to the formation of viral capsid or viral-capsid like structure with an inherent repetitive organization, wherein said structure is, typically, spherical or tubular.

One common feature of virus particle and virus-like particle is its highly ordered and repetitive arrangement of its subunits.

Virus-like particle of a RNA bacteriophage: As used herein, the term “virus-like particle of a RNA bacteriophage” refers to a virus-like particle comprising, or preferably consisting essentially of or consisting of coat proteins, mutants or fragments thereof, of a RNA bacteriophage. In addition, virus-like particle of a RNA bacteriophage resembling the structure of a RNA bacteriophage, being non replicative and/or non-infectious, and lacking at least the gene or genes encoding for the replication machinery of the RNA bacteriophage, and typically also lacking the gene or genes encoding the protein or proteins responsible for viral attachment to or entry into the host. This definition should, however, also encompass virus-like particles of RNA bacteriophages, in which the aforementioned gene or genes are still present but inactive, and, therefore, also leading to non-replicative and/or non-infectious virus-like particles of a RNA bacteriophage. Within this present disclosure the term “subunit” and “monomer” are interexchangeably and equivalently used within this context.

One, a, or an: when the terms “one”, “a”, or “an” are used in this disclosure, they mean “at least one” or “one or more” unless otherwise indicated.

Within this application, antibodies are defined to be specifically binding if they bind to the antigen with a binding affinity (Ka) of 10⁶ M⁻¹ or greater, preferably 10⁷ M⁻¹ or greater, more preferably 10⁸ M⁻¹ or greater, and most preferably 10⁹ M⁻¹ or greater. The affinity of an antibody can be readily determined by one of ordinary skill in the art (for example, by Scatchard analysis.)

The amino acid sequence identity of polypeptides can be determined conventionally using known computer programs such as the Bestfit program. When using Bestfit or any other sequence alignment program, preferably using Bestfit, to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, polypeptides or a fragment thereof disclosed in this invention.

Conservative amino acid substitutions, as understood by a skilled person in the art, include isosteric substitutions, substitutions where the charged, polar, aromatic, aliphatic or hydrophobic nature of the amino acid is maintained. Typical conservative amino acid substitutions are substitutions between amino acids within one of the following groups: Gly, Ala; Val, Ile, Leu; Asp, Glu; Asn, Gln; Ser, Thr, Cys; Lys, Arg; and Phe and Tyr.

This invention provides compositions and methods for enhancing immune responses against antigen of the invention in an animal or in human. Composition of the invention comprises: (a) a virus-like particle (VLP) with at least one first attachment site; and (b) at least one antigen with at least one second attachment site, wherein the at least one antigen is an antigen of the invention and wherein (a) and (b) are linked through the at least one first and the at least one second attachment site. Preferably, the antigen of the invention is linked to the VLP, so as to form an ordered and repetitive antigen-VLP array. In preferred embodiments of the invention, at least 20, preferably at least 30, more preferably at least 60, again more preferably at least 120 and further more preferably at least 180 antigen of the invention are linked to the VLP.

Any virus known in the art having an ordered and repetitive structure may be selected as a VLP of the invention. Illustrative DNA or RNA viruses, the coat or capsid protein of which can be used for the preparation of VLPs have been disclosed in WO 2004/009124 on page 25, line 10-21, on page 26, line 11-28, and on page 28, line 4 to page 31, line 4. These disclosures are incorporated herein by way of reference.

Virus or virus-like particle can be produced and purified from virus-infected cell culture. The resulting virus or virus-like particle for vaccine purpose needs to be devoid of virulence. Besides genetic engineering, physical or chemical methods can be employed to inactivate the viral genome function, such as UV irradiation, formaldehyde treatment.

In one preferred embodiment, the VLP is a recombinant VLP. Almost all commonly known viruses have been sequenced and are readily available to the public. The gene encoding the coat protein can be easily identified by a skilled artisan. The preparation of VLPs by recombinantly expressing the coat protein in a host is within the common knowledge of a skilled artisan.

In one preferred embodiment, the virus-like particle comprises, or alternatively consists of, recombinant proteins, mutants or fragments thereof, of a virus selected form the group consisting of: a) RNA phages; b) bacteriophages; c) Hepatitis B virus, preferably its capsid protein (Ulrich, et al., Virus Res. 50:141-182 (1998)) or its surface protein (WO 92/11291); d) measles virus (Warnes, et al., Gene 160:173-178 (1995)); e) Sindbis virus; f) rotavirus (U.S. Pat. No. 5,071,651 and U.S. Pat. No. 5,374,426); g) foot-and-mouth-disease virus (Twomey, et al., Vaccine 13:1603 1610, (1995)); h) Norwalk virus (Jiang, X., et al., Science 250:1580 1583 (1990); Matsui, S. M., et al., J. Clin. Invest. 87:1456 1461 (1991)); i) Alphavirus; j) retrovirus, preferably its GAG protein (WO 96/30523); k) retrotransposon Ty, preferably the protein p1; 1) human Papilloma virus (WO 98/15631); m) Polyoma virus; n) Tobacco mosaic virus; and o) Flock House Virus.

In one preferred embodiment, the VLP comprises, or consists of, more than one amino acid sequence, preferably two amino acid sequences, of the recombinant proteins, mutants or fragments thereof. VLP comprises or consists of more than one amino acid sequence is referred, in this application, as mosaic VLP.

The term “fragment of a recombinant protein” or the term “fragment of a coat protein”, as used herein, is defined as a polypeptide, which is of at least 70%, preferably at least 80%, more preferably at least 90%, even more preferably at least 95% the length of the wild-type recombinant protein, or coat protein, respectively and which preferably retains the capability of forming VLP. Preferably the fragment is obtained by at least one internal deletion, at least one truncation or at least one combination thereof. The term “fragment of a recombinant protein” or “fragment of a coat protein” shall further encompass polypeptide, which has at least 80%, preferably 90%, even more preferably 95% amino acid sequence identity with the “fragment of a recombinant protein” or “fragment of a coat protein”, respectively, as defined above and which is preferably capable of assembling into a virus-like particle.

The term “mutant recombinant protein” or the term “mutant of a recombinant protein” as interchangeably used in this invention, or the term “mutant coat protein” or the term “mutant of a coat protein”, as interchangeably used in this invention, refers to a polypeptide having an amino acid sequence derived from the wild type recombinant protein, or coat protein, respectively, wherein the amino acid sequence is at least 80%, preferably at least 85%, 90%, 95%, 97%, or 99% identical to the wild type sequence and preferably retains the ability to assemble into a VLP.

In one preferred embodiment, the virus-like particle of the invention is of Hepatitis B virus. The preparation of Hepatitis B virus-like particles have been disclosed, inter alia, in WO 00/32227, WO 01/85208 and in WO 01/056905. All three documents are explicitly incorporated herein by way of reference. Other variants of HBcAg suitable for use in the practice of the present invention have been disclosed in page 34-39 WO 01/056905.

In one further preferred embodiments of the invention, a lysine residue is introduced into the HBcAg polypeptide, to mediate the linking of antigen of the invention to the VLP of HBcAg. In preferred embodiments, VLPs and compositions of the invention are prepared using a HBcAg comprising, or alternatively consisting of, amino acids 1-144, or 1-149, 1-185 of SEQ ID NO:20, which is modified so that the amino acids at positions 79 and 80 are replaced with a peptide having the amino acid sequence of Gly-Gly-Lys-Gly-Gly. This modification changes the SEQ ID NO:20 to SEQ ID NO:21. In further preferred embodiments, the cysteine residues at positions 48 and 110 of SEQ ID NO:21, or its corresponding fragments, preferably 1-144 or 1-149, are mutated to serine. The invention further includes compositions comprising Hepatitis B core protein mutants having above noted corresponding amino acid alterations. The invention further includes compositions and vaccines, respectively, comprising HBcAg polypeptides which comprise, or alternatively consist of, amino acid sequences which are at least 80%, 85%, 90%, 95%, 97% or 99% identical to SEQ ID NO:21.

In one preferred embodiment, the virus-like particle is of a Cowpea Chlorotic Mottle Virus, a Cowpea Mosaic Virus or an Alfalfa Mosaic Virus. Methods to produce VLP of these viruses have been described in US 2005/0260758 and in WO05067478.

In preferred embodiments of the invention, the virus-like particle is of an RNA bacteriophage. Preferably, the RNA-bacteriophage is selected from the group consisting of a) bacteriophage Qβ; b) bacteriophage R17; c) bacteriophage fr; d) bacteriophage GA; e) bacteriophage SP; f) bacteriophage MS2; g) bacteriophage M11; h) bacteriophage MX1; i) bacteriophage NL95; k) bacteriophage f2; l) bacteriophage PP7 and m) bacteriophage AP205.

In one preferred embodiment of the invention, the composition comprises coat protein, mutants or fragments thereof, of RNA bacteriophages, wherein the coat protein has amino acid sequence selected from the group consisting of: (a) SEQ ID NO:1, referring to Qβ CP; (b) a mixture of SEQ ID NO:1 and SEQ ID NO:2.(referring to Qβ A1 protein); (c) SEQ ID NO:3; (d) SEQ ID NO:4; (e) SEQ ID NO:5; (f) SEQ ID NO:6, (g) a mixture of SEQ ID NO:6 and SEQ ID NO:7; (h) SEQ ID NO:8; (i) SEQ ID NO:9; (j) SEQ ID NO:10; (k) SEQ ID NO:11; (1) SEQ ID NO:12; (m) SEQ ID NO:13; and (n) SEQ ID NO:14. Generally the coat protein mentioned above is capable of assembly into VLP with or without the presence of the N-terminal methionine.

In one preferred embodiment of the invention, the VLP is a mosaic VLP comprising or alternatively consisting of more than one amino acid sequence, preferably two amino acid sequences, of coat proteins, mutants or fragments thereof, of a RNA phage.

In one very preferred embodiment, the VLP comprises or alternatively consists of two different coat proteins of a RNA phage, said two coat proteins have an amino acid sequence of SEQ ID NO: 1 and SEQ ID NO:2, or of SEQ ID NO:6 and SEQ ID NO:7.

In preferred embodiments of the present invention, the virus-like particle of the invention comprises, or alternatively consists essentially of, or alternatively consists of recombinant coat proteins, mutants or fragments thereof, of the RNA-bacteriophage Qβ, fr, AP205 or GA.

In one preferred embodiment, the VLP of the invention is a VLP of RNA-phage Qβ. Further preferred virus-like particles of RNA-phages, in particular of Qβ and fr in accordance of this invention are disclosed in WO 02/056905, the disclosure of which is herewith incorporated by reference in its entirety. Particular example 18 of WO 02/056905 gave detailed description of preparation of VLP particles from Qβ.

In another preferred embodiment, the VLP of the invention is a VLP of RNA phage AP205. Assembly-competent mutant forms of AP205 VLPs, including AP205 coat protein with the substitution of proline at amino acid 5 to threonine, asparigine at amino acid 14 to aspartic acid, may also be used in the practice of the invention and leads to other preferred embodiments of the invention. WO 2004/007538 describes, in particular in Example 1 and Example 2, how to obtain VLP comprising AP205 coat proteins, and hereby in particular the expression and the purification thereto. WO 2004/007538 is incorporated herein by way of reference.

In one preferred embodiment, the VLP of the invention comprises or consists of a mutant coat protein of a virus, preferably a RNA phage, wherein the mutant coat protein has been modified by removal of at least one lysine residue by way of substitution and/or by way of deletion. In another preferred embodiment, the VLP of the invention comprises or consists of a mutant coat protein of a virus, preferably a RNA phage, wherein the mutant coat protein has been modified by addition of at least one lysine residue by way of substitution and/or by way of insertion. In one very preferred embodiment, the mutant coat protein is of RNA phage Qβ, wherein at least one, or alternatively at least two, lysine residue have been removed by way of substitution or by way of deletion. In an alternative very preferred embodiment, the mutant coat protein is of RNA phage Qβ, wherein at least one, or alternatively at least two, lysine residue have been added by way of substitution or by way of insertion. In one further preferred embodiment, the mutant coat protein of RNA phage Qβ has an amino acid sequence selected from any one of SEQ ID NO:15-19.

In one preferred embodiment, the compositions and vaccines of the invention have an antigen density being from 0.5, preferably from 1.0, preferably from 1.2, preferably from 1.6, preferably from 1.9, preferably from 2.2 to 4.0. The term “antigen density”, as used herein, refers to the average number of antigen of the invention which is linked per subunit, preferably per coat protein, of the VLP, and hereby preferably of the VLP of a RNA phage. Thus, this value is calculated as an average over all the subunits or monomers of the VLP, preferably of the VLP of the RNA-phage, in the composition or vaccines of the invention.

Further RNA phage coat proteins have also been shown to self-assemble upon expression in a bacterial host (Kastelein, R A. et al., Gene 23:245-254 (1983), Kozlovskaya, T M. et al., Dokl. Akad. Nauk SSSR 287:452-455 (1986), Adhin, M R. et al., Virology 170:238-242 (1989), Priano, C. et al., J. Mol. Biol. 249:283-297 (1995)). In particular the biological and biochemical properties of GA (Ni, C Z., et al., Protein Sci. 5:2485-2493 (1996), Tars, K et al., J. Mol. Biol. 271:759-773 (1997)) and of fr (Pushko P. et al., Prot. Eng. 6:883-891 (1993), Liljas, L et al. J. Mol. Biol. 244:279-290, (1994)) have been disclosed. The crystal structure of several RNA bacteriophages has been determined (Golmohammadi, R. et al., Structure 4:543-554 (1996)). Using such information, surface exposed residues can be identified and, thus, RNA-phage coat proteins can be modified such that one or more reactive amino acid residues can be inserted by way of insertion or substitution. Another advantage of the VLPs derived from RNA phages is their high expression yield in bacteria that allows production of large quantities of material at affordable cost.

In one preferred embodiment, the antigen of the invention is a CCR5 extracellular domain, a CCR5 extracellular domain fragment or any combination thereof. In one preferred embodiment of the invention, the at least one antigen is a CCR5 extracellular domain fragment. In one preferred embodiment, the CCR5 extracellular domain fragment comprises, consists essentially of or consists of a CCR5 extracellular domain ECL2 fragment, preferably ECL2A. ECL2A, as generally understood in the art, starts preferably from the first amino acid from the N-terminus of the ECL2 and stops preferably at threonine, which is right before cysteine in the human ECL2 sequence. In one further preferred embodiment, the CCR5 extracellular domain fragment comprises, consists essentially of or alternatively consists of SEQ ID NO: 25. In one preferred embodiment, the CCR5 extracellular domain fragment comprises, consists essentially of or consists of a cyclized ECL2A. In a further preferred embodiment, the CCR5 extracellular domain fragment comprises, consists essentially of, or alternatively consists of the cyclized SEQ ID NO:25. In a further preferred embodiment, the CCR5 extracellular domain fragment comprises, consists essentially of or alternatively consists of the cyclized SEQ ID NO:26 or SEQ ID NO:52, wherein the peptide is cyclized by the C and G residue at both ends. Cyclized SEQ ID NO:25, as used herein, refers to an amino acid sequence comprising, consisting essentially of or consisting of SEQ ID NO.25, wherein the first amino acid residue and the last amino acid residue of said amino acid sequence interact with each other by at least one chemical bond, preferably by at least one covalent bond. Preferably the first amino acid residue and the last amino acid residue of said amino acid sequence interact with each other by all covalent bonds. Preferably the first amino acid residue and the last amino acid residue of said amino acid sequence interact with each other by one peptide bond, leading to a circular peptide.

In one preferred embodiment of the invention, the at least one antigen is a CCR5 extracellular domain PNt. In one further preferred embodiment, the CCR5 extracellular domain PNt comprises, consists essentially of or alternatively consists of SEQ ID NO:27. In one further preferred embodiment, the CCR5 extracellular domain PNt comprises, or consists of SEQ ID NO:27 with additional linker, preferably cysteine, fused to either the C— or the N-terminus of SEQ ID NO:27, preferably fused to the C-terminus of SEQ ID NO:27. In still further preferred embodiment, the CCR5 extracellular domain PNt comprises, consists essentially of, or consists of SEQ ID NO:27 with additional linker, preferably cysteine, fused to either the C— or the N-terminus of SEQ ID NO:27, preferably fused to the C-terminus of SEQ ID NO:27, wherein the naturally occurring cysteine within SEQ ID NO:27 was substituted by another amino acid, preferably by serine. This is to ensure a uniform and defined antigen presentation.

In one preferred embodiment, the present invention provides a composition comprising: (a) a virus-like particle (VLP) of an RNA-bacteriophage with at least two first attachment sites; and (b) at least one CCR5 extracellular domain PNt with at least two second attachment sites; wherein said CCR5 extracellular domain PNt comprises, consists essentially of or consists of: (i) a Nta domain or a Nta domain fragment, and (ii) a Ntb domain comprising amino acids 23 to 27 of SEQ ID NO:27 (SEQ ID NO:56) or Ntb domain fragment comprising amino acids 23 to 27 of SEQ ID NO:27, wherein the C-terminus of said Nta domain or said Nta domain fragment is fused, preferably directly, to said N-terminus of said Ntb domain or said Ntb domain fragment, and wherein the first or the second of said at least two second attachment sites comprises or is a sulfhydryl group, preferably a sulfhydryl group of a cysteine residue, and wherein the first of said at least two second attachment sites is located upstream of the N-terminus of said amino acids 23 to 27 of SEQ ID NO:27; and wherein the second of said at least two second attachment sites is located downstream of the C terminus of said amino acids 23 to 27 of SEQ ID NO:27, preferably downstream of the C terminus of said CCR5 extracellular domain PNt; and wherein said VLP of said RNA-bacteriophage and said CCR5 extracellular domain PNt are linked by at least one non-peptide covalent bond. Nta domain: the term “Nta domain” as used herein, refers to the native Nta domain having the amino acid sequence of SEQ ID NO:57 or the corresponding sequence of CCR5 orthologs from any other animals, preferably from primates (including anthropoidea and prosimii), more preferably from anthropoidea. Furthermore, the term “Nta domain”, as used herein, refers to a modified Nta domain, in which three, preferably two, more preferably one amino acid of the native Nta domain, as defined herein, has been modified by deletion, insertion and/or substitution, preferably conservative substitution, with the proviso that antibodies elicited by the inventive compositions comprising said modified Nta domain bind specifically to human CCR5.

Nta domain fragment: the term “Nta domain fragment” as used herein, refers to any polypeptide comprises, consists essentially of or consists of at least 8, preferably at least 9, 10, 11, 12, 13, 14, 15 or 16 consecutive amino acid sequence of the Nta domain as defined herein, with the proviso that antibodies elicited by the inventive compositions comprising said Nta domain fragment bind specifically to human CCR5.

Ntb domain: the term “Ntb domain” as used herein, refers to the native Ntb domain having the amino acid sequence of SEQ ID NO:58 or the corresponding sequence of CCR5 orthologs from any other animals, preferably from primates (including anthropoidea and prosimii), more preferably from anthropoidea. Furthermore, the term “Ntb domain”, as used herein, refers to a modified Ntb domain, in which two, preferably one amino acid of the native Ntb domain, as defined herein, has been modified by deletion, insertion and/or substitution, preferably by conservative substitution, with the proviso that antibodies elicited by the inventive compositions comprising said modified Ntb domain bind specifically to human CCR5.

Ntb domain fragment: the term “Ntb domain fragment” as used herein, refers to any polypeptide comprises, consists essentially of or consists of at least 6, preferably at least 7, 8, 9, 10 consecutive amino acid sequence of the Ntb domain as defined herein, with the proviso that antibodies elicited by the inventive compositions comprising said Ntb domain fragment bind specifically to human CCR5. Preferably said Ntb domain fragment comprises, consists essentially of, or consists of amino acid sequence CQKINVK (SEQ ID NO:59), more preferably CQKINVKQ (SEQ ID NO:60). Furthermore, said Ntb domain fragment comprises, consists essentially of, or consists of amino acid sequence CQKINVK, more preferably CQKINVKQ, in which one amino acid of CQKINVK or CQKINVKQ has been modified by deletion, insertion and/or substitution, preferably conservative substitution, with the proviso that antibodies elicited by the inventive compositions comprising said Ntb domain fragment bind specifically to human CCR5.

In one preferred embodiment, the CCR5 extracellular domain PNt with at least two second attachment sites does not comprise a further sulfhydryl group of cysteine, preferably a further sulfhydryl group, beside said two sulfhydryl groups, preferably two sulfhydryl groups of said cysteine residues, comprised by or being said first and said second of said at least two second attachment sites.

In one preferred embodiment, the first of said at least two second attachment sites is not located upstream of the N-terminus of the Nta domain or the Nta domain fragment. This is to ensure the free access of the N-terminus of the Nta domain or the Nta domain fragment to the host immune system since the natural configuration of CCR5 has a free moving N-terminus. Preferably the first of said at least two second attachment sites is located downstream of the C-terminus of the Nta domain or the Nta domain fragment.

In one preferred embodiment, the first of said at least two second attachment sites is the naturally occurring cysteine residue within said CCR5 extracellular domain PNt. In one preferred embodiment, the first of said at least two second attachment sites corresponds to the sulfhydryl group of the cysteine residue of SEQ ID NO:27.

In one alternative embodiment, the first of said at least two second attachment sites is located one, two or three amino acid position(s) upstream, or one or two amino acid position(s) downstream of said naturally occurring cysteine, wherein preferably said first of said at least two second attachment sites has been generated by insertion or by substitution of the naturally occurring amino acid residue at that position into cysteine; and wherein preferably said naturally occurring cysteine within PNt domain has been deleted or substituted, preferably by a serine or an alanine substitution.

In one preferred embodiment, the CCR5 extracellular domain PNt comprises, consists essentially of, or preferably consists of the amino acid sequence of SEQ ID NO:27. In one preferred embodiment, the CCR5 extracellular domain PNt comprises, consists essentially of, or preferably consists of the amino acid sequence derived from SEQ ID NO:27, in which three, preferably two, preferably one amino acid of SEQ ID NO:27 has been modified by insertion, deletion and/or substitution, preferably conservative substitution, with the proviso that antibodies elicited by the inventive compositions comprising said amino acid sequence derived from SEQ ID NO:27 bind specifically to human CCR5.

In one preferred embodiment, the composition further comprises a linker, wherein said linker is fused to the C-terminus of said CCR5 extracellular domain PNt, and wherein said linker comprises or is the second of said at least two second attachment sites. The linker can be of varied length so that the flexibility of Ntb domain or Ntb domain fragment can be adjusted for more efficient coupling to different VLPs or for better mimicking the natural configuration of the native Ntb domain.

In one preferred embodiment, the linker is selected from the group consisting of: (a) GGC; (b) GGC-CONH2; (c) GC; (d) GC-CONH2; (e) C; and (f) C—CONH2. In one further preferred embodiment, the linker is a cysteine or an amidated cysteine. In one preferred embodiment, the CCR5 extracellular domain PNt with at least two second attachment sites comprises, consists essentially of, or preferably consists of the amino acid sequence of SEQ ID NO:44.

In one preferred embodiment, the first and the second of said at least two second attachment sites associate with the at least two first attachment sites through at least two non-peptide covalent bonds. In one further preferred embodiment, only the first and the second of said at least two second attachment sites associate with the at least two first attachment sites through at least two non-peptide covalent bonds, typically and preferably leading to a “bridge-like” structure of the Ntb domain or Ntb domain fragment. Without being bound by the proposed theory, it is believed that this bridge-like structure mimics the natural configuration of the native Ntb domain, the N-terminus of which is engaged in a disulfide bond with another cysteine in the ECL3 loop, and the C-terminus of which is anchored to the cell membrane.

In one preferred embodiment, the at least two first attachment sites comprise identical reactive functionality. Preferably each of the at least two first attachment sites comprises an amino group. More preferably each of the at least two first attachment sites comprises an amino group of a lysine residue.

In one preferred embodiment, the composition further comprises at least two hetero-bifunctional molecules, wherein said at least two hetero-bifunctional molecules link said at least two first attachment sites and said at least two second attachment sites, wherein preferably each of said at least two hetero-bifunctional molecules is SMPH.

In one preferred embodiment, the virus-like particle of RNA-bacteriophage is Qβ, AP205, fr or GA. In one preferred embodiment, the virus-like particle of RNA-bacteriophage is Qβ. At least four lysine residues are exposed on the surface of the VLP of Qβ coat protein. This lysine density ensures that one of the at least two second attachment sites quickly finds and links the first attachment site after the other one of the at least two second attachment sites has linked one first attachment site by at least one non-peptide covalent bond. Similarly VLPs of other RNA-bacteriophages are also suitable for this invention.

In one aspect, this invention provides a method of producing a composition comprising the steps of: (a) providing a virus-like particle of an RNA-bacteriophage with at least two first attachment sites; wherein said virus-like particle (VLP) of an RNA-bacteriophage comprises or consists of coat proteins, mutants or fragments thereof, of said RNA-bacteriophage; wherein preferably each of said at least two first attachment sites comprises or is an amino group, preferably an amino group of a lysine residue; (b) providing at least one CCR5 extracellular domain PNt with at least two second attachment sites; wherein said CCR5 extracellular domain PNt comprises, consists essentially of or consists of: (i) a Nta domain or a Nta domain fragment, and (ii) a Ntb domain comprising amino acids 23 to 27 of SEQ ID NO:27 (SEQ ID NO:56) or Ntb domain fragment comprising amino acids 23 to 27 of SEQ ID NO:27, wherein the C-terminus of said Nta domain or said Nta domain fragment is fused, preferably directly, to said N-terminus of said Ntb domain or said Ntb domain fragment, and wherein the first or the second of said at least two second attachment sites comprises or is a sulfhydryl group, preferably a sulfhydryl group of a cysteine residue, and wherein the first of said at least two second attachment sites is located upstream of the N-terminus of said amino acids 23 to 27 of SEQ ID NO:27; and wherein the second of said at least two second attachment sites is located downstream of the C terminus of said amino acids 23 to 27 of SEQ ID NO:27, preferably downstream of the C terminus of said CCR5 extracellular domain PNt; (c) linking the VLP of said RNA-bacteriophage and the CCR5 extracellular domain PNt by at least one non-peptide covalent bond.

In one preferred embodiment, the molecular ratio of the CCR5 extracellular domain PNt to the coat protein of the VLP of an RNA bacteriophage is from 8:1 to 0.5:1, preferably from 4:1, to 1:1, more preferably from 4:1 to 2:1, still more preferably 2:1.

In one preferred embodiment, the step (a) further comprises adding to said virus-like particle (VLP) of RNA-bacteriophage a heterobiofunctional linker, wherein preferably said heterobiofunctional linker is SMPH. Preferably the molecular ratio of SMPH to coat protein of the VLP of RNA bacteriophage is from 40:1 to 2:1, preferably from 20:1 to 4:1, more preferably 10:1.

in one preferred embodiment, linking said VLP of RNA bacteriophage and said CCR5 extracellular domain PNt site is carried out in a solution with ion strength not more than 150 mM, preferably not more than 100 mM, preferably not more than 75, more preferably not more than 50 mM.

In one preferred embodiment, the virus-like particle of RNA-bacteriophage is Qβ, AP205, fr or GA, preferably Qβ.

In one preferred embodiment, the CCR5 extracellular domain PNt comprises, consists essentially of, or preferably consists of the amino acid sequence of SEQ ID NO:27. In one preferred embodiment, the CCR5 extracellular domain PNt comprises, consists essentially of, or preferably consists of the amino acid sequence derived from SEQ ID NO:27, in which three, preferably two, preferably one amino acid of SEQ ID NO:27 has been modified by insertion, deletion and/or substitution, preferably conservative substitution with the proviso that antibodies elicited by the inventive compositions comprising said amino acid sequence derived from SEQ ID NO:27 bind specifically to human CCR5.

In one preferred embodiment, the CCR5 extracellular domain PNt with at least two second attachment sites comprises, consists essentially of, or preferably consists of the amino acid sequence of SEQ ID NO:44.

In one aspect, the invention provides a composition obtainable or preferably obtained according to the method of the invention.

In one preferred embodiment, the antigen of the invention is a CXCR4 extracellular domain, a CXCR4 extracellular domain fragment or any combination thereof. In one preferred embodiment, the CXCR4 extracellular domain is the CXCR4 N-terminal extracellular domain. In one preferred embodiment, the CXCR4 N-terminal extracellular domain is coupled via its C-terminus to the virus-like particle.

In one preferred embodiment, the CXCR4 N-terminal extracellular domain comprises, consists essentially of or consists of SEQ ID NO:30 or an amino acid sequence derived from SEQ ID NO:30, in which three, preferably two, preferably one amino acid of SEQ ID NO:30 has been modified by insertion, deletion and/or substitution, preferably conservative substitution with the proviso that antibodies elicited by the inventive compositions comprising said amino acid sequence derived from SEQ ID NO:30 bind specifically to human CXCR4. In one further preferred embodiment, the CXCR4 N-terminal extracellular domain comprising, consisting essentially of or consisting of SEQ ID NO:30 is coupled via its C-terminus to the virus-like particle.

In one preferred embodiment, the CXCR4 extracellular domain fragment is CXCR4 extracellular domain ECL2 fragment. In a further preferred embodiment, the CXCR4 extracellular ECL2 domain fragment comprises, consists essentially of, or consists of SEQ ID NO:29 or an amino acid sequence derived from SEQ ID NO:29, in which two, preferably one amino acid of SEQ ID NO:29 has been modified by insertion, deletion and/or substitution, preferably conservative substitution, with the proviso that antibodies elicited by the inventive compositions comprising said amino acid sequence derived from SEQ ID NO:29 bind specifically to human CXCR4. In one preferred embodiment, the CXCR4 extracellular ECL2 domain fragment comprises, consists essentially of or consists essentially of, or consists of liner, i.e. non-cyclized SEQ ID NO:29 or said amino acid sequence derived from SEQ ID NO:29. In one further preferred embodiment, said liner SEQ ID NO:29 is coupled to the virus-like particle, either via its N-terminus or C-terminus, preferably via its C-terminus.

In one preferred embodiment, the CXCR4 extracellular domain fragment comprises or consists of cyclized CXCR4 extracellular ECL2 domain fragment. In a further preferred embodiment, the CXCR4 extracellular domain fragment comprises, consists essentially of or alternatively consists of cyclized SEQ ID NO:29 or an cyclized amino acid sequence derived from SEQ ID NO:29. Cyclized SEQ ID NO:29, as used herein, refers to an amino acid sequence comprising or consisting of SEQ ID NO.29, wherein the first amino acid residue and the last amino acid residue of said amino acid sequence interact with each other by at least one chemical bond, preferably by at least one covalent bond. Preferably the first amino acid residue and the last amino acid residue of said amino acid sequence interact with each other by all covalent bonds. Preferably the first amino acid residue and the last amino acid residue of said amino acid sequence interact with each other by one peptide bond, leading to a circular peptide. In a further preferred embodiment, the CXCR4 extracellular ECL2 domain fragment comprises or consists of cyclized SEQ ID NO:49 or SEQ ID NO:53, wherein the peptide is cyclized by the C and G residue at both ends.

In one preferred embodiment, the at least one antigen is gastrin of the invention. In one embodiment, the at least one antigen is gastrin G17. In one preferred embodiment, the gastrin G17 comprises, consists essentially of or consists of SEQ ID NO:34. In one further preferred embodiment, the gastrin G17 comprises, consists essentially of, or consists of SEQ ID NO:36. In one alternative further preferred embodiment, the gastrin G17 comprises, consists essentially of or preferably consists of SEQ ID NO:34 with the last amino acid F being amidated.

In one preferred embodiment, the at least one antigen is progastrin G34. In one preferred embodiment, the progastrin G34 comprises, consists essentially of or consists of SEQ ID NO:35. In one further preferred embodiment, the progastrin G34 comprises or consists of SEQ ID NO:37. In one alternative further preferred embodiment, the progastrin G34 comprises, consists essentially of or consists of SEQ ID NO:35 with the last amino acid F being amidated.

In one preferred embodiment, the at least one antigen comprises, consists essentially of or consists of gastrin G17 1-9 fragment (SEQ ID NO:33), preferably with a linker sequence fused to its C-terminus, more preferably with a linker sequence SSPPPPC fused to the C-terminus (SEQ ID NO:39).

In one very preferred embodiment, the gastrin of the invention fused with a linker comprises, consists essentially of or consists of SEQ ID NO:38.

In one preferred embodiment, the gastrin of the invention with at least one second attachment site comprises, consists essentially of, or alternatively consists of an amino acid sequence selected from the group consisting of SEQ ID NO:38; SEQ ID NO:39; SEQ ID NO:40; SEQ ID NO:41; SEQ ID NO:42; and SEQ ID NO:43.

It is to note E at position one of sequence EGPWLEEEE as part of gastrin sequence could be E, pyro E or Q. When additional amino acid is fused to the N-terminus of EGPWLEEEE, E at position one of sequence EGPWLEEEE could be E or preferably Q.

In one preferred embodiment, the at least one antigen of the invention is a CETP fragment. In one further preferred embodiment, the CETP fragment comprises, consists essentially of, or consists of a polypeptide having amino acid sequence as SEQ ID NO:32 or a polypeptide derived from SEQ ID NO:32, in which two, preferably one amino acid of SEQ ID NO:32 has been modified by insertion, deletion and/or substitution, preferably conservative substitution with the proviso that antibodies elicited by the inventive compositions comprising said polypeptide derived from SEQ ID NO:32 bind specifically to CETP, preferably human CETP.

In one preferred embodiment, the at least one antigen is a C5a protein. In one preferred embodiment, the C5a protein comprises, consists essentially of or consists of a polypeptide having amino acid sequence as SEQ ID NO:45 or a polypeptide derived from SEQ ID NO:45, in which five, four, preferably three, preferably two, preferably one amino acid of SEQ ID NO:45 has been modified by insertion, deletion and/or substitution, preferably conservative substitution with the proviso that antibodies elicited by the inventive compositions comprising said polypeptide derived from SEQ ID NO:45 bind specifically to C5a, preferably human C5a. In one preferred embodiment, the at least one antigen is a C5a fragment. In one further preferred embodiment, the C5a fragment comprises, consists essentially of, or consists of a polypeptide having amino acid sequence as SEQ ID NO:46 or a polypeptide derived from SEQ ID NO:46, in which two, preferably one amino acid of SEQ ID NO:46 has been modified by insertion, deletion and/or substitution, preferably conservative substitution with the proviso that antibodies elicited by the inventive compositions comprising said polypeptide derived from SEQ ID NO:46 bind specifically to C5a, preferably human C5a.

In one preferred embodiment, the antigen of the invention is a Bradykinin of the invention. Bradykinin (BK, KRPPGFSPFR, SEQ ID NO:50) is a major vasodilator peptide and plays an important role in the local regulation of blood pressure, blood flow and vascular permeability (Margolies H. S, et al., Hypertension, 1995). Bradykinin exerts its effects via the B2-receptor.

des-Arg9-BK (KRPPGFSPF, SEQ ID NO:51) has overlapping and distinct functions from Bradykinin. Evidence suggests that des-Arg9-BK is rapidly generated after tissue injury and modulates most of the events observed during inflammatory processes including vasodilatation, increase of vascular permeability, plasma extravasation, cell migration, pain and hyperalgesia (Calixto J. B. et al., Pain 2000). Des-Arg9-BK exerts its effects via the B1-receptor

BK and Des-Arg9-BK have been reported to play a role in several inflammatory diseases (Cruwys S. C. et al., Br J Pharmacol, 1994; Cassim B. et al., Immunopharmacology 1997). Experimental evidence suggests that both BK des-Arg9-BK play a role during the development of asthma (Christiansen S. C. et al., Am. Rev. Dis. 1992; Barnes P. J. et al., Thorax, 1992); Fuller R. W. et al., Am. Rev. Respir. Dis., 1987).

In one further preferred embodiment, the Bradykinin of the invention further comprises a linker fused to the N-terminus of the Bradykinin of the invention, preferably the linker sequence is a cysteine. In one further preferred embodiment, the Bradykinin of the invention further comprises a linker fused to the C-terminal of the Bradykinin of the invention, preferably the linker sequence is a cysteine. In one further preferred embodiment, the Bradykinin of the invention comprises or consists of SEQ ID NO:50.

In one preferred embodiment, the antigen of the invention is a des-Arg-Bradykinin of the invention. In one further preferred embodiment, the composition of the invention further comprises a linker fused to the N-terminus of des-Arg-Bradykinin of the invention, preferably the linker sequence is a cysteine. In one further preferred embodiment, the composition of the invention further comprises a linker fused to the C-terminal of des-Arg-Bradykinin of the invention, preferably the linker sequence is a cysteine. In one further preferred embodiment, the des-Arg-Bradykinin of the invention comprises or consists of SEQ ID NO:51.

In yet another preferred embodiment, the at least one antigen comprises or alternatively consists of at least one antigenic site of the antigen of the invention.

It is known that possession of immunogenicity does not usually require the full length of a protein and usually a protein contains more than one antigenic epitope, i.e. antigenic site. A fragment or a short peptide may be sufficient to contain at least one antigenic site that can be bound immunospecifically by an antibody or by a T-cell receptor within the context of an MHC molecule. Antigenic site or sites can be determined by a number of techniques generally known to the skilled person in the art. Methods to determine antigenic site(s) of a protein is known to the skilled person in the art. WO2005/108425 has elaborated some of these methods from paragraph [0099] to [0103] and these specific disclosures are incorporated herein by way of reference. It is to note that these methods are generally applicable to other polypeptide antigens, and therefore not restricted to IL-23 p19 as disclosed in WO2005/108425.

In one preferred embodiment of the invention, the VLP with at least one first attachment site is linked to the antigen of the invention with at least one second attachment site via at least one peptide bond. Gene encoding antigen of the invention, preferably antigen of the invention not longer than 75 amino acids, preferably not longer than 50 amino acids, even more preferably less than 30 amino acids, is in-frame ligated, either internally or preferably to the N- or the C-terminus to the gene encoding the coat protein of the VLP. Fusion may also be effected by inserting sequences of the antigen of the invention into a mutant of a coat protein where part of the coat protein sequence has been deleted, that are further referred to as truncation mutants. Truncation mutants may have N- or C-terminal, or internal deletions of part of the sequence of the coat protein. For example for the specific VLP HBcAg, amino acids 79-80 are replaced with a foreign epitope. The fusion protein shall preferably retain the ability of assembly into a VLP upon expression which can be examined by electromicroscopy.

Flanking amino acid residues may be added to increase the distance between the coat protein and foreign epitope. Glycine and serine residues are particularly favored amino acids to be used in the flanking sequences. Such a flanking sequence confers additional flexibility, which may diminish the potential destabilizing effect of fusing a foreign sequence into the sequence of a VLP subunit and diminish the interference with the assembly by the presence of the foreign epitope.

In one preferred embodiment, the modified VLP is a mosaic VLP, wherein preferably said mosaic VLP comprises or alternatively consists of at least one fusion protein and at least one viral coat protein.

In other embodiments, the at least one antigen of the invention, preferably the antigen of the invention consisting of less than 50 amino acids can be fused to a number of other viral coat protein, as way of examples, to the C-terminus of a truncated form of the A1 protein of Qβ (Kozlovska, T. M., et al., Intervirology 39:9-15 (1996)), or being inserted between position 72 and 73 of the CP extension. For example, Kozlovska et al., (Intervirology, 39: 9-15 (1996)) describe QβA1 protein fusions where the epitope is fused at the C-terminus of the QβCP extension truncated at position 19. As another example, the antigen of the invention can be inserted between amino acid 2 and 3 of the fr CP (Pushko P. et al., Prot. Eng. 6:883-891 (1993)). Furthermore, the antigen of the invention can be fused to the N-terminal protuberant β-hairpin of the coat protein of RNA phage MS-2 (WO 92/13081). Alternatively, the antigen of the invention can be fused to a capsid protein of papillomavirus, preferably to the major capsid protein L1 of bovine papillomavirus type 1 (BPV-1) (Chackerian, B. et al., Proc. Natl. Acad. Sci. USA 96:2373-2378 (1999), WO 00/23955). Substitution of amino acids 130-136 of BPV-1 L1 with an antigen of the invention is also an embodiment of the invention. Further embodiments o fusing antigen of the invention to coat protein, mutants or fragements thereof, to a coat protein of a virus have been disclosed in WO 2004/009124 page 62 line 20 to page 68 line 17 and herein are incorporated by way of reference.

In another preferred embodiment, the at least one antigen of the invention, preferably the antigen of the invention composed of less than 70 amino acids, preferably with less than 50 amino acids is fused to either the N- or the C-terminus of a coat protein, mutants or fragments thereof, of RNA phage AP205. In one further preferred embodiment, the fusion protein further comprises a spacer, wherein said spacer is fused to the coat protein, fragments or mutants thereof, of AP205 and the antigen of the invention. Preferably said spacer composed of less than 30, preferably less than 20, even more preferably less than 10, still more preferably less than 5 amino acids.

In one preferred embodiment of the present invention, the composition comprises or alternatively consists essentially of a virus-like particle with at least one first attachment site linked to at least one antigen of the invention with at least one second attachment site via at least one covalent bond, preferably the covalent bond is a non-peptide bond. Preferably the first attachment site does not comprise or is not sulfhydryl group of cysteine. Further preferably the first attachment site does not comprise or is not sulfhydryl group. In a preferred embodiment of the present invention, the first attachment site comprises, or preferably is, an amino group, preferably the amino group of a lysine residue. In another preferred embodiment of the present invention, the second attachment site comprises, or preferably is, a sulfhydryl group, preferably a sulfhydryl group of a cysteine.

In a very preferred embodiment or the invention, at least one first attachment site comprises, or preferably is, an amino group, preferably an amino group of a lysine residue and the at least one second attachment site comprises, or preferably is, a sulfhydryl group, preferably a sulfhydryl group of a cysteine residue.

In one preferred embodiment of the invention, the antigen of the invention is linked to the VLP by way of chemical cross-linking, typically and preferably by using a heterobifunctional cross-linker. In preferred embodiments, the hetero-bifunctional cross-linker contains a functional group which can react with the preferred first attachment sites, preferably with the amino group, more preferably with the amino groups of lysine residue(s) of the VLP, and a further functional group which can react with the preferred second attachment site, i.e. a sulfhydryl group, preferably of cysteine(s) residue inherent of, or artificially added to the antigen of the invention, and optionally also made available for reaction by reduction. Several hetero-bifunctional cross-linkers are known to the art. These include the preferred cross-linkers SMPH (Pierce), Sulfo-MBS, Sulfo-EMCS, Sulfo-GMBS, Sulfo-SIAB, Sulfo-SMPB, Sulfo-SMCC, SVSB, SIA and other cross-linkers available for example from the Pierce Chemical Company, and having one functional group reactive towards amino groups and one functional group reactive towards sulfhydryl groups. The above mentioned cross-linkers all lead to formation of an amide bond after reaction with the amino group and a thioether linkage with the sulfhydryl groups. Another class of cross-linkers suitable in the practice of the invention is characterized by the introduction of a disulfide linkage between the antigen of the invention and the VLP upon coupling. Preferred cross-linkers belonging to this class include, for example, SPDP and Sulfo-LC-SPDP (Pierce).

In a preferred embodiment, the composition of the invention further comprises a linker. Engineering of a second attachment site onto the antigen of the invention is achieved by the association of a linker, preferably containing at least one amino acid suitable as second attachment site according to the disclosures of this invention. Therefore, in a preferred embodiment of the present invention, a linker is associated to the antigen of the invention by way of at least one covalent bond, preferably, by at least one, typically one peptide bond. Preferably, the linker comprises, or alternatively consists of, the second attachment site. In a further preferred embodiment, the linker comprises a sulfhydryl group, preferably of a cysteine residue. In another preferred embodiment, the amino acid linker is a cysteine residue.

The selection of linkers has been disclosed in WO2005/108425A1, page 32-33, which is incorporated herein by way of reference.

Linking of the antigen of the invention to the VLP by using a hetero-bifunctional cross-linker according to the preferred methods described above, allows coupling of the antigen of the invention to the VLP in an oriented fashion. Other methods of linking the antigen of the invention to the VLP include methods wherein the antigen of the invention is cross-linked to the VLP, using the carbodiimide EDC, and NHS. The antigen of the invention may also be first thiolated through reaction, for example with SATA, SATP or iminothiolane. The antigen of the invention, after deprotection if required, may then be coupled to the VLP as follows. After separation of the excess thiolation reagent, the antigen of the invention is reacted with the VLP, previously activated with a hetero-bifunctional cross-linker comprising a cysteine reactive moiety, and therefore displaying at least one or several functional groups reactive towards cysteine residues, to which the thiolated antigen of the invention can react, such as described above. Optionally, low amounts of a reducing agent are included in the reaction mixture. In further methods, the antigen of the invention is attached to the VLP, using a homo-bifunctional cross-linker such as glutaraldehyde, DSG, BM[PEO]4, BS3, (Pierce) or other known homo-bifunctional cross-linkers with functional groups reactive towards amine groups or carboxyl groups of the VLP.

In other embodiments of the present invention, the composition comprises or alternatively consists essentially of a virus-like particle linked to antigen of the invention via chemical interactions, wherein at least one of these interactions is not a covalent bond. Such interactions include but not limited to antigen-antibody interaction, receptor-ligand interaction. Linking of the VLP to the antigen of the invention can be effected by biotinylating the VLP and expressing the antigen of the invention as a streptavidin-fusion protein.

In one preferred embodiment of the invention, the VLP of the invention is recombinantly produced by a host and wherein said VLP is essentially free of host RNA, preferably host nucleic acids. In one further preferred embodiment, the composition further comprises at least one polyanionic macromolecule bound to, preferably packaged in or enclosed in, the VLP. In a still further preferred embodiment, the polyanionic macromolecule is polyglutamic acid and/or polyaspartic acid.

Essentially free of host RNA, preferably host nucleic acids: The term “essentially free of host RNA, preferably host nucleic acids” as used herein, refers to the amount of host RNA, preferably host nucleic acids, comprised by the VLP, which amount typically and preferably is less than 30 μg, preferably less than 20 μg, more preferably less than 10 μg, even more preferably less than 8 μg, even more preferably less than 6 μg, even more preferably less than 4 μg, most preferably less than 2 per mg of the VLP. Host, as used within the aforementioned context, refers to the host in which the VLP is recombinantly produced. Conventional methods of determining the amount of RNA, preferably nucleic acids, are known to the skilled person in the art. The typical and preferred method to determine the amount of RNA, preferably nucleic acids, in accordance with the present invention is described in Example 17 of WO2006/037787A2 filed on Oct. 5, 2005 by the same applicant. Identical, similar or analogous conditions are, typically and preferably, used for the determination of the amount of RNA, preferably nucleic acids, for inventive compositions comprising VLPs other than Qβ. The modifications of the conditions eventually needed are within the knowledge of the skilled person in the art. The numeric value of the amounts determined should typically and preferably be understood as comprising values having a deviation of ±10%, preferably having a deviation of ±5%, of the indicated numeric value.

Polyanionic macromolecule: The term “polyanionic macromolecule”, as used herein, refers to a molecule of high relative molecular mass which comprises repetitive groups of negative charge, the structure of which essentially comprises the multiple repetition of units derived, actually or conceptually, from molecules of low relative molecular mass. A polyanionic macromolecule should have a molecular weight of at least 2000 Dalton, more preferably of at least 3000 Dalton and even more preferably of at least 5000 Dalton. The term “polyanionic macromolecule” as used herein, typically and preferably refers to a molecule that is not capable of activating toll-like receptors. Thus, the term “polyanionic macromolecule” typically and preferably excludes Toll-like receptors ligands, and even more preferably furthermore excludes immunostimulatory substances such as Toll-like receptors ligands, immunostimulatory nucleic acids, and lipopolysacchrides (LPS). More preferably the term “polyanionic macromolecule” as used herein, refers to a molecule that is not capable of inducing cytokine production. Even more preferably the term “polyanionic macromolecule” excludes immunostimulatory substances. The term “immunostimulatory substance”, as used herein, refers to a molecule that is capable of inducing and/or enhancing immune response specifically against the antigen comprised in the present invention.

Host RNA, preferably host nucleic acids: The term “host RNA, preferably host nucleic acids” or the term “host RNA, preferably host nucleic acids, with secondary structure”, as used herein, refers to the RNA, or preferably nucleic acids, that are originally synthesized by the host. The RNA, preferably nucleic acids, may, however, undergo chemical and/or physical changes during the procedure of reducing or eliminating the amount of RNA, preferably nucleic acids, typically and preferably by way of the inventive methods, for example, the size of the RNA, preferably nucleic acids, may be shortened or the secondary structure thereof may be altered. However, even such resulting RNA or nucleic acids is still considered as host RNA, or host nucleic acids.

Methods to determine the amount of RNA and to reduce the amount of RNA comprised by the VLP have been disclosed in WO2006/037787A2 filed by the same applicant on Oct. 5, 2005 and thus the entire application, in particular examples 4, 5 and 17, are incorporated herein by way of reference. Reducing or eliminating the amount of host RNA, preferably host nucleic, minimizes or reduces unwanted T cell responses, such as inflammatory T cell response and cytotoxic T cell response, and other unwanted side effects, such as fever, while maintaining strong antibody response specifically against the antigen.

In one aspect, the invention provides a vaccine composition comprising, consistings essentially of, or consisting of the composition of the invention. In one preferred embodiment, the antigen of the invention linked to the VLP in the vaccine composition may be of animal, preferably mammal or human origin. In preferred embodiments, the antigen of the invention is of human, bovine, dog, cat, mouse, rat, pig or horse origin.

In one preferred embodiment, the vaccine composition further comprises at least one adjuvant. The administration of the at least one adjuvant may hereby occur prior to, contemporaneously or after the administration of the inventive composition. The term “adjuvant” as used herein refers to non-specific stimulators of the immune response or substances that allow generation of a depot in the host which when combined with the vaccine and pharmaceutical composition, respectively, of the present invention may provide for an even more enhanced immune response.

In another preferred embodiment, the vaccine composition is devoid of adjuvant. An advantageous feature of the present invention is the high immunogenicity of the composition, even in the absence of adjuvants. The absence of an adjuvant, furthermore, minimizes the occurrence of unwanted inflammatory T-cell responses representing a safety concern in the vaccination against self antigens. Thus, the administration of the vaccine of the invention to a patient will preferably occur without administering at least one adjuvant to the same patient prior to, contemporaneously or after the administration of the vaccine. VLP has been generally described as an adjuvant. However, the term “adjuvant”, as used within the context of this application, refers to an adjuvant not being the VLP used for the inventive compositions, rather in addition to said VLP.

The invention further discloses a method of immunization comprising administering the vaccine of the present invention to an animal or a human. The animal is preferably a mammal, such as cat, sheep, pig, horse, bovine, dog, rat, mouse and particularly human. The vaccine may be administered to an animal or a human by various methods known in the art, but will normally be administered by injection, infusion, inhalation, oral administration, or other suitable physical methods. The conjugates may alternatively be administered intramuscularly, intravenously, transmucosally, transdermally, intranasally, intraperitoneally or subcutaneously. Components of conjugates for administration include sterile aqueous (e.g., physiological saline) or non-aqueous solutions and suspensions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption.

Vaccines of the invention are said to be “pharmacologically acceptable” if their administration can be tolerated by a recipient individual. Further, the vaccines of the invention will be administered in a “therapeutically effective amount” (i.e., an amount that produces a desired physiological effect). The nature or type of immune response is not a limiting factor of this disclosure. Without the intention to limit the present invention by the following mechanistic explanation, the inventive vaccine might induce antibodies which bind to CCR5, CXCR4, gastrin, progastrin, CETP, C5a, Bradykinin or des-Arg-Bradykinin and thus reducing its concentration and/or interfering with its physiological or pathological function.

In one aspect, the invention provides a pharmaceutical composition comprising, consists essentially of, or consisting of the composition as taught in the present invention and an acceptable pharmaceutical carrier. When vaccine of the invention is administered to an individual, it may be in a form which contains salts, buffers, adjuvants, or other substances which are desirable for improving the efficacy of the conjugate. Examples of materials suitable for use in preparation of pharmaceutical compositions are provided in numerous sources including REMINGTON'S PHARMACEUTICAL SCIENCES (Osol, A, ed., Mack Publishing Co., (1990)).

The invention teaches a process for producing the composition of the invention comprising the steps of: (a) providing a VLP with at least one first attachment site; (b) providing a antigen of the invention with at least one second attachment site, and (c) combining said VLP and said antigen of the invention to produce a composition, wherein said antigen of the invention and said VLP are linked through the first and the second attachment sites. In a preferred embodiment, the provision of the at least one antigen of the invention, with the at least one second attachment site is by way of expression, preferably by way of expression in a bacterial system, preferably in E. coli. Usually tag, such as His tag, Myc tag is added to facilitate the purification process. In another approach particularly the at least one antigen of the invention with no longer than 50 amino acids can be chemically synthesized.

In a further preferred embodiment, the step of providing a VLP with at least one first attachment site comprises further steps: (a) disassembling said virus-like particle to said coat proteins, mutants or fragments thereof, of said RNA-bacteriophage; (b) purifying said coat proteins, mutants or fragments thereof; (c) reassembling said purified coat proteins, mutants or fragments thereof, of said RNA-bacteriophage to a virus-like particle, wherein said virus-like particle is essentially free of host RNA, preferably host nucleic acids. In a still further preferred embodiment, the reassembling of said purified coat proteins is effected in the presence of at least one polyanionic macromolecule.

In one preferred embodiment, the present invention provides a method of preventing and/or treating HIV infection, wherein the method comprises administering the inventive composition or the inventive vaccine composition, respectively, to a human, wherein the antigen of the invention is a CCR5 of the invention.

In one preferred embodiment, the present invention provides a method of preventing and/or treating HIV infection, wherein the method comprises administering the inventive composition or the inventive vaccine composition, respectively, to a human, wherein the antigen of the invention is a CXCR4 of the invention.

In one preferred embodiment, the present invention provides a method of preventing and/or treating atheroslerosis, wherein the method comprises administering the inventive composition or the inventive vaccine composition, respectively, to an animal or a human, wherein the antigen of the invention is a CETP of the invention. Atherosclerosis is an arterial disease that includes but is not limited to coronary heart disease, coronary artery disease, carotid artery disease and cerebrovascular disease.

In one preferred embodiment, the present invention provides a method of preventing and/or treating primary and/or chronic inflammatory diseases, wherein the method comprises administering the inventive composition or the invention vaccine composition, respectively, to an animal or a human, wherein the antigen of the invention is a C5a of the invention, Primary and/or chronic inflammatory diseases, in which C5a mediates or contributes to the condition, include but are not limited to rheumatoid arthritis, systemic lupus erythematosus. asthma and bullous pemphigoid.

In one preferred embodiment, the present invention provides a method of preventing and/or treating primary and/or chronic inflammatory diseases, wherein the method comprises administering the inventive composition or the inventive vaccine composition, respectively, to an animal or a human, wherein the antigen of the invention is a Bradykinin of the invention. Primary and/or chronic inflammatory diseases, in which Bradykinin mediates or contributes to the condition, include but not are limited to arthritis and asthma.

In one preferred embodiment, the present invention provides a method of preventing and/or treating primary and/or chronic inflammatory diseases, wherein the method comprises administering the inventive composition or the inventive vaccine composition, respectively, to an animal or a human, wherein the antigen of the invention is a des-Arg-Bradykinin of the invention. Primary and/or chronic inflammatory diseases, in which des-Arg-Bradykinin mediates or contributes to the condition, include but are not limited to arthritis and asthma.

In one preferred embodiment, the present invention provides a method of preventing and/or treating cancer, in particular cancers of gastrointestinal tract, wherein the method comprises administering the inventive composition or the inventive vaccine composition, respectively, to an animal or a human, wherein the antigen of the invention is a gastrin of the invention. Cancers of gastrointestinal tract include but are not limited to gastric carcinoma, colon cancer, rectal cancer and pancreatic cancer.

In another aspect, the invention provides the composition of the invention for use as a medicament, wherein the antigen of the invention is CCR5 of the invention, CXCR4 of the invention, gastrin of the invention, CETP of the invention, C5a of the invention, Bradykinin of the invention or des-Arg-Bradykinin of the invention, respectively.

In one preferred embodiment, the invention provides for the use of the composition for the manufacture of a medicament for prevention and/or treatment of HIV infection in human, wherein said composition comprises at least one CCR5 of the invention.

In one preferred embodiment, the invention provides for the use of the composition for the manufacture of a medicament for prevention and/or treatment of HIV infection in human, wherein said composition comprises at least one CXCR4 of the invention.

In one preferred embodiment, the invention provides for the use of the composition for the manufacture of a medicament for prevention and/or treatment of atheroslerosis, wherein said composition comprises at least one CETP of the invention. Atherosclerosis is an arterial disease that includes but is not limited to coronary heart disease, coronary artery disease, carotid artery disease and cerebrovascular disease.

In one preferred embodiment, the present invention provides for the use of the composition for the manufacture of a medicament for prevention and/or treatment of primary and/or chronic inflammatory diseases, wherein said composition comprising at least one C5a of the invention. Primary and/or chronic inflammatory diseases, in which C5a mediates or contributes to the condition, include but are not limited to rheumatoid arthritis, systemic lupus erythematosus. asthma and bullous pemphigoid.

In one preferred embodiment, the present invention provides for the use of the composition for the manufacture of a medicament for prevention and/or treatment primary and/or chronic inflammatory diseases, wherein said composition comprising at least one Bradykinin of the invention. Primary and/or chronic inflammatory diseases, in which Bradykinin mediates or contributes to the condition, include but not are limited to arthritis and asthma.

In one preferred embodiment, the present invention provides for the use of the composition for the manufacture of a medicament for prevention and/or treatment primary and/or chronic inflammatory diseases, wherein said composition comprising at least one des-Arg-Bradykinin of the invention. Primary and/or chronic inflammatory diseases, in which des-Arg-Bradykinin mediates or contributes to the condition, include but are not limited to arthritis and asthma.

In one preferred embodiment, the present invention provides for the use of the composition for the manufacture of a medicament for prevention and/or treatment cancer, in particular cancers of gastrointestinal tract, wherein said composition comprising at least one gastrin of the invention. Cancers of gastrointestinal tract include but are not limited to gastric carcinoma, colon cancer, rectal cancer and pancreatic cancer.

### EXAMPLES

### Example 1

### Coupling of CCR5PNt Peptides or ECL2A to Qβ VLP

2 g/l Qβ VLPs (143 μM of Qβ coat protein) were derivatised with 1.43 mM SMPH (Pierce) for 30 minutes at 25° C. and then dialysed against 20 mM Hepes pH8, 150 mM NaCl. 0.286 mM peptide PNt-CC (SEQ ID NO:44, from 3 mM stock in DMSO) with the C-terminus cysteine amidated and 1 g/l derivatised Qβ particles were incubated for two hours at 25° C.

As second method, 2 g/l Qβ VLPs were derivatised with 1.43 mM SMPH for 30 minutes at 25° C. and then dialysed against 20 mM phosphate pH 7.4. 0.143 mM peptide PNt-CC (SEQ ID NO:44, from 50 mM stock in DMSO) with the C-terminus cysteine amidated and 1 g/l derivatised Qβ particles were incubated for two hours at 25° C. The coupling product was dialysed against 20 mM Phosphate pH 7.4.

2 g/l Qβ were derivatised with 1.43 mM SMPH for 30 minutes at 25° C. and then dialysed against 20 mM Hepes pH 7.4, 150 mM NaCl. 0.286 mM peptide PNt-SC (SEQ ID NO:54, from 5 mM stock in DMSO) with the C-terminus cysteine amidated and 1 g/l derivatised Qβ particles were incubated for two hours at 25° C. The coupling product was dialysed against 20 mM Hepes pH 7.4, 150 mM NaCl.

2 g/l Qβ were derivatised with 1.43 mM SMPH for 30 minutes at 25° C. and then dialysed against 20 mM Hepes pH 7.4, 150 mM NaCl. 0.143 mM peptide PNt-CN (SEQ ID NO:55, from 5 mM stock in DMSO) with the C-terminus cysteine amidated and 1 g/l derivatised Qβ particles were incubated for two hours at 25° C. The coupling product was dialysed against 20 mM Hepes pH 7.4, 150 mM NaCl.

2 g/l Qβ were derivatised with 1.43 mM SMPH for 30 minutes at 25° C. and then dialysed against 20 mM phosphate pH 7.5. 1 g/l derivatised Qβ particles were dissolved in 20% acetonitrile and 0.286 mM cyclic ECL2A (SEQ ID NO:26, from a 5 mM stock solution in DMSO) were added and incubated for two hours at 25° C. in 20 mM phosphate pH 7.5, 150 mM NaCl. The coupling product was dialysed against 20 mM phosphate pH 7.5.

### Example 2

### Immunisation

C57BL/6 mice were primed with 50 μg Qβ-PNtCC, Qβ-PNtCN, Qβ-PNtSC or Qβ-ECL2A (obtained from EXAMPLE 1) on day 0, (subcutaneously, in 0.2 ml 20 mM phosphate pH 7.5) and compared to Balb/C mice primed with 50 μg Qβ only. After boosting with the same vaccines on day 14, the α-Qβ and the α-CCR5 peptide antibody titers were checked by ELISA at day 14 and day 21 (TABLE 1).

TABLE 1

| Constructs | ELISA titer |
|---|---|
| PNt-CC | 4802 |
| ECL2A | 4698 |

Alternatively, New Zealand White rabbits were primed with 100 μg Qβ-PNtCC (obtained from EXAMPLE 1, second method) on day 0, (intradermic at 10 points on the back of the rabbit) with equal parts (v/v) of complete Freund's adjuvant. The following three boosts (100 μg Qβ-PNtCC on days 14, 28, 56) were carried out with equal parts (v/v) incomplete Freund's adjuvant. The α-Qβ and the α-CCR5 peptide antibody titers were checked by ELISA at day 37 and day 56, and found to be always above 12'000.

### Example 3

### Purification of Polyclonal Mouse or Rabbit IgG

Sera pooled from five Qβ-PNtCC, Qβ-PNtCN, Qβ-PNtSC or Qβ-ECL2A immunised mice, respectively, (or two rabbits) (obtained from EXAMPLE 4) were centrifuged for five minutes at 14'000 rpm. The supernatant was loaded on a column of 3.3 ml prewashed protein G sepharose (Amersham). The column was then washed with PBS and eluted with 100 mM glycine pH 2.8. 1 ml fractions were collected in tubes previously provided with 112 μl 1 M Tris pH8. Peak fractions absorbing at 280 nm were pooled.

### Example 4

### Affinity Purification of Polyclonal Rabbit IgG

1 mg of Qβ or Qβ-PNtCC was immobilized on an N-hydroxysuccinimide activated Sepharose column, according to the manufacturers instructions (GE Healthcare Europe). 5 mg rabbit IgG (from EXAMPLE 3) was loaded in PBS on a Qβ affinity column with a flow rate of 0.5 ml/min. The flow-through fraction was collected for further PNtCC specific purification. specific IgG were eluted from the Qβ column with 100 mM glycine pH 2.6 and neutralized with 120 mM Tris pH 8. PNtCC specific IgG in the flow-through fraction were further purified on a Qβ-PNtCC column. Eluted and neutralized IgG were washed 4 times with PBS using a centrifugal filter device (Amicon Ultra-4, 10'000 MWCO).

### Example 5

### FACS Staining of Cellular CCR5 with Mouse Polyclonal IgG

CEM.NKR-CCR5 is a CCR5-expressing variant of the CEM.NKR cell line, a human line that naturally expresses CD4 (Trkola et al., J. Virol., 1999, page 8966). CEM.NKR-CCR5 cells were grown in RPMI 1640 culture medium (with 10% FCS, glutamine, and antibiotics). Cells were pelleted and resuspended in phosphate-buffered saline (PBS) containing 1% fetal calf serum (FCS) in order to get 2.3×10⁶ cells/ml. A [1:250] dilution of human IgG (Miltenyi Biotec) was added as a blocking agent and incubated for 20 minutes. The cells were washed once in 1% FCS/PBS and 0.1 ml (2.3×10⁵ cells/well) were plated and incubated with CCR5 polyclonal antibodies purified from EXAMPLE 3 (60 mg/l; eluted from protein G column; dilutions with 1% FCS/PBS). After 30 minutes at 4° C., the cells were washed once in 1% FCS/PBS and stained for 20 minutes at 4° C. with 15 mg/l FITC-goat-α-mouse-IgG (Jackson) in 1% FCS/PBS. After two washes in 1% FCS/PBS, 5′000-10′000 stained cells were analysed by flow cytometry. The geometric mean of each staining was determined using the “cell quest” flow cytometry software.

TABLE 2 shows that PNtCC or ECL2A specific antibodies specifically bind to CCR5 molecules expressed on the cell surface of CEM.NKR, whereas the PNtSC, and PNtCN specific antibodies as well as the Qβ specific antibodies do not bind to CCR5 molecules expressed on the cell surface.

TABLE 2

| Polyclonal purified total IgG | Geometric mean (FL-1H) |
|---|---|
| PNtCC | 29.3 |
| PNtSC | 8.4 |
| PNtCN | 9.8 |
| ECL2A | 15.8 |
| Qβ | 6.4 |
| No IgG | 4.6 |

### Example 6

### HIV-Neutralisation Assay

Briefly, buffy coats obtained from 3 healthy blood donors were depleted of CD8+ T cells using Rosette Sep cocktail (StemCell Technologies Inc) and PBMC were isolated by Ficoll-Hypaque centrifugation (Amersham-Pharmacia Biotech). Cells were adjusted to 4×10⁶/ml in culture medium (RPMI 1640, 10% FCS, 100 U/ml IL-2, glutamine and antibiotics), divided into three parts and stimulated with either 5 μg/ml phytohemagglutinin (PHA), 0.5 μg/ml PHA or 1 mg/l anti-CD3 MAb OKT3. After 72 h, cells from all three stimulations were combined and used as source of stimulated CD4+T cells for infection and virus neutralisation experiments.

HIV neutralisation assay was performed essentially as described previously (Trkola et al., J. Virol., 1999, page 8966). The R5 viruses (CCR5 co-receptor specific strains), JR-FL and SF162, have been described previously (O'Brien et al., Nature 1990, 348, page 69; and Shioda et al., Nature 1991, 349, page 167). Briefly, cells were incubated with serial dilutions of purified polyclonal rabbit IgG (25 μg/ml-25 ng/ml, obtained from EXAMPLE 5 or EXAMPLE 6) or positive control HIV-inhibitor Rantes in 96-well culture plates for 1 h at 37° C.

The HIV-1 inoculums were adjusted to contain approximately 1,000 to 4,000 TCID₅₀/ml in assay medium (TCID₅₀: 50% tissue culture infective dose, Trkola et al., J. Virol., 1999, page 8966). Virus inoculum (100 TCID₅₀; 50% tissue culture infective dose) was added and plates cultured for 7 days. The total infection volume was 200; A. Then, the supernatant medium was assayed for the HIV-1 p24 antigen production by using an immunoassay, as described previously (Moore et al., 1990. Science 250, page 1139).

TABLE 3 shows that the purified antibodies efficiently neutralize HIV up to 70% at a low antibody concentration (e.g. 0.56 μg/ml).

TABLE 3
Antibody concentrations inhibiting HIV

| | 50% inhibition | 70% inhibition | |
|---|---|---|---|
| Qβ affinity purified | >25 | >25 | μg/ml |
| PNtCC affinity purified | 0.23 | 0.56 | μg/ml |
| PNtCC total IgG | 0.36 | 1.01 | μg/ml |
| RANTES | 5.8 | 14.7 | ng/ml |

HIV Neutralization Assay with CEM 5.25 Cells

Neutralization activity of purified mouse serum immunoglobulin samples against the virus isolate JR-FL was evaluated on CEM 5.25.EGFP.luc.M7 cells (Nathaniel Landau) using JR-FL envelope pseudotyped luciferase reporter virus as described (Montefiori, D.C. (2004). Evaluating neutralizing antibodies against HIV, SW and SHIV in luciferase reporter gene assays. Current Protocols in Immunology, John Wiley & Sons, 12.11.1-12.11.15. and Wei, X., et al, Nature 422:307-12). CEM 5.25.EGFP.luc.M7 cells were incubated with serial dilutions of mouse antibodies (obtained from EXAMPLE 3) for 1 h at 37° C. Virus inoculum (150 TCID₅₀) and polybrene (final concentration 10 ng/ml) was then added. The total infection volume was 200 μl. The Ig concentration causing 50% reduction (NT₅₀) in luciferase reporter gene production after 72 h was determined by regression analysis.

TABLE 4 shows that PNtCC specific total IgG inhibited HIV infection at low concentration, whereas PNtCN specific IgG did not inhibit HIV infection at any measured concentration.

TABLE 4
Antibody concentrations inhibiting HIV

| | 50% inhibition | |
|---|---|---|
| PNt-CN | >25 | μg/ml |
| PNt-CC | 1.45 | μg/ml |
| Positive control | 0.17 | μg/ml |
| (Mab to CCR5) | | |

### Example 7

### In-Gel Digestion and LC/MS Analysis of Qβ-PNtCC

Samples of Qβ-3-PNtCC, Qβ-PNtSC and derivatized Qβ (obtained from EXAMPLE 1) were loaded on a reducing SDS-PAGE gel. Gel bands corresponding to the Qβ monomer per peptide and Qβ dimer per peptide (or Qβ monomer and Qβ dimer in the case of derivatized Qβ) were cut in small pieces and washed twice with 100 μl 100 mM NH₄HCO₃, 50% acetonitrile, and washed once with 50 μl acetonitrile. All three supernatants were discarded. Then, 10 μl protease Glu-C (0.01 ng/μl in 10 mM Tris, pH 8.2) and 10 μl buffer (10 mM Tris, pH 8.2) were added and incubated at 37° C. overnight. The supernatant was stored and gel pieces were extracted twice with 100 μl 0.1% trifluoroacetic acid, 50% acetonitrile. All three supernatants were combined and dried. The samples were dissolved in 15 μl 0.1% formic acid. 6 μl was injected onto the HPLC column and masses of the peptides were determined by LC/MS.

### Example 8

### Chemical Synthesis of CXCR4 Fragments (aa1-39) and (aa176-185) and Coupling to Qβ VLP

CXCR4 fragment 1-39 (SEQ ID NO:30) with a CGG or GGC linker sequence fused to either the N- or the C-terminus of the CXCR4 fragment 1-39, CXCR4 fragment 176-185 (SEQ ID NO:29) with a CGG or GGC linker fused at either the N- or the C-terminus or CXCR4 fragment 176-185 (SEQ ID NO:29) which was cyclized by connecting a C which was added at the N-terminus with a G which was added at the C-terminus were chemically synthesized according to standard procedures (Peter Henklein, Charité).

A solution of 3 ml (1.0 mg/ml) Qβ VLP in 20 mM Hepes, pH 7.2 was reacted for 30 minutes with 85 μl SMPH (50 mM in DMSO, Pierce) at 25° C. The dialysed, derivatized Qβ VLP was subsequently used to couple peptides CXCR4-CGG-1-39, CXCR4—1-39-GGC, CXCR4-CGG-176-185, CXCR4—176-185-GGC or CXCR4-C-176-185-G. Briefly, 1 ml of derivatized Qβ VLP at a concentration of 1 mg/ml was reacted with 70 μl of a 5 mM peptide solution for 2 hours at 25° C. in 20 mM Hepes, pH 7.2.

The coupling efficiency was estimated to be between 0.24-0.5 CXCR4 fragments per Qβ monomer.

### Example 9

### Immunization of Mice with CXCR4 Fragments

Adult female, C57BL/6 mice (3 per group) were vaccinated with Qβ-CXCR4-fragments (obtained in EXAMPLE 8), using Qβ VLP as a control. 100 μg of dialyzed vaccine from each sample were diluted in PBS to a volume of 200 μl and injected subcutaneously (100 μl on two ventral sides) on days 0 and 14. The vaccines were administered without or with adjuvant (Allhydrogel, 1 mg/injection). Mice were bled retro-orbitally on day 14, 21, 28 and peptide-specific antibody responses were determined by ELISA by coating CXCR4-peptides coupled to RNase at a concentration of 10 μg/ml in coating buffer (0.1 M NaHCO₃, pH 9.6), over night at 4° C. CXCR4 was coupled to RNase Briefly as the following: 5 mg/ml RNase was derivatized in 0.2 mM SPDP (SIGMA) for 1 h at RT. Derivatized RNase solution was then purified over a PD10 column (Amersham). 10 mM EDTA and 1 mM peptide were added to the derivatized RNase solution and the reaction was incubated for 1 h.

TABLE 5

| | ELISA titer | | | |
|---|---|---|---|---|
| | without ALUM | | with ALUM | |
| Constructs | d 14 | d 28 | d 14 | d 28 |
| CXCR4-CGG-1-39-VLP | 13493 | 30719 | 14201 | 40959 |
| CXCR4-1-39-GGC-VLP | 11167 | 30719 | 3337 | 40959 |
| CXCR4-CGG-176-185-VLP | 86 | 1279 | 40 | 920 |
| CXCR4-176-185-GGC-VLP | 240 | 1759 | 388 | 28159 |
| CXCR4-C-176-185-G-VLP | 13837 | 40960 | 9049 | 40960 |

Mean peptide specific ELISA titers in sera of three mice per group are shown.

### Example 10

### Detection of CXCR4-Specific Antibodies by Surface Staining of the Human T-Cell Lines Jurkat and CEM.NKR-CCR5

Jurkat cells or CEM.NKR-CCR5 cells were grown in RPMI 1640 culture medium supplemented with 10% FCS, glutamine, and antibiotics. Cells were harvested, washed and resuspended in phosphate-buffered saline (PBS) containing 1% fetal calf serum (FCS). To prevent Fe-receptor mediated binding, cells were first incubated for 30 min with rat-α-mouse-CD16/CD32 (BD Pharmingen) in PBS/1% FCS for at 4° C. After washing the cells (1×10⁵) were incubated with serially diluted mouse serum (obtained from EXAMPLE 10) for 30 min at 4° C. Cells were washed with PBS/1% FCS and incubated with FITC-α-mouse-IgG (BD Pharmingen) for 30 min at 4° C., cells were then analysed with a FACS Calibur and specific binding of the antibodies was quantified by using CellQuest software (BD Biosciences). The results are summarized in TABLE below.

TABLE 6

| | Mean Fluoresence intensity | | | |
|---|---|---|---|---|
| | CEM.NKR-CCR5 | | Jurkat | |
| | cells | | cells | |
| | | d 21/ | | d 21/ |
| Construct | d 21* | ALUM* | d 21* | ALUM* |
| CXCR4-CGG-1-39-VLP | 96.7 | 98.1 | 43.2 | 48 |
| CXCR4-1-39-GGC-VLP | 97.2 | 307.1 | 57.5 | 71.4 |
| CXCR4-CGG-176-185-VLP | 57.2 | 60.9 | 33.7 | 42.3 |
| CXCR4-176-185-GGC-VLP | 75.7 | 109.5 | 45.0 | 108.7 |
| CXCR4-C-176-185-G-VLP | 76.6 | 128.12 | 83.8 | 74.7 |
| VLP (Control) | 51 | 51 | 32 | 32 |

*Serum of d 21 after first immunization at a dilution of 1:200 was used for the staining of the cells.

### Example 11

### R4HIV-1 Strain Neutralization Assay

Briefly, buffy coats obtained from 3 healthy blood donors are first depleted of CD8+T cells using Rosette Sep cocktail (StemCell Technologies Inc., BIOCOBA AG) and peripheral blood mononuclear cells are collected by Ficoll-Hypaque centrifugation (Amersham-Pharmacia Biotech). Purified cells are then adjusted to 4×10⁶/ml in culture medium (RPMI 1640, 10% FCS, 100 U/ml IL-2, glutamine and antibiotics), divided into three samples and stimulated with either 5 μg/ml phytohemagglutinin (PHA), 0.5 μg/ml PHA or 1 mg/l anti-CD3 MAb OKT3. After 72 h, the cells are combined and used as stimulated CD4+T cells for infection and virus neutralisation experiments.

To test the neutralizing potential, cells are first incubated with serial dilutions of purified polyclonal mouse IgG (as described above) or control antibody 12G5 (25 μg/ml±25 ng/ml; Pharmingen) in 96-well culture plates for 1 h at 37° C.

The X4 strains NL4-3 and 2044 have been described previously (Trkola et al (1998), J. Virol. 72:396; Trkoly et al (1998), J. Virol 72-1876). Then virus inoculum (100 TCID₅₀; 50% tissue culture infective dose; Trkola et al., J. Virol., 1999, page 8966) are added and the cells are cultured for another 4-14 days. The total infection volume is 200 μd. On day 6 post infection, the supernatant are assayed for the amount of HIV-1 p24 antigen production by using an immunoassay, as described previously (Moore et al., 1990. Science 250, page 1139).

### Example 12

### Coupling of CETP Fragment to Qβ VLP

The CETP peptide CETP1, having the carboxy-terminal sequence ranging from amino acid 461-476 (SEQ ID NO:32) of human CETP and fused at its N-terminus with the tripeptide CGG for coupling to VLPs was synthesized by solid phase chemistry at EMC microcollections GmbH. The peptide was amidated at its C-terminus.

A solution of 750 μl (4.0 mg/ml) VLP in 20 mM Hepes, 150 mM NaCl pH 7.4 was reacted for 30 minutes with a 10-fold excess of SMPH (21.4 μl of a 100 mM stock in DMSO, Pierce) at 25° C. 1.5 ml of derivatized Qβ VLP at a concentration of 2 mg/ml was reacted with 21 μl of a 50 mM CETP peptide solution for 2 hours at 15° C. in 20 mM Hepes, 150 nM NaCl, pH 7.4.

### Example 13

### Immunization of Mice with Qβ-CETP1 and ELISA

Female Balb/c mice (n=3) were vaccinated with CETP1 coupled to Qβ VLP. 50 μg of dialyzed vaccine were diluted in PBS to a volume of 200 μl and injected subcutaneously (100 μl on two ventral sides) on day 0, 14, 50 and 73. The vaccine was administered without adjuvant. Antibody titers were determined in the sera of the mice bled retro-orbitally on day 0, 70 and 80.

CETP1 was coupled to AP205 VLP (20 mM Hepes, 150 mM NaCl pH 7.4) for coating to ELISA plates. Briefly, 1 ml of 1 mg/ml AP205 VLP was derivatized with 7.1 μl of a 50 mM SMPH (Pierce) stock (in DMSO) for 30 minutes at RT. Derivatized AP205 solution (1 ml) was reacted with 7.1 μl of a 50 mM stock of CETP1 (in DMSO), and incubated for 2 h at 15° C. CETP1 was also coupled to BSA for coating to ELISA plates.

ELISA plates were coated with CETP peptide coupled to AP205 VLP or BSA at a concentration of 5 μg/ml in coating buffer (0.1 M NaHCO₃, pH 9.6), over night at 4° C.

TABLE 7 Average anti-CETP 1 specific IgG antibody titer (expressed as the reciprocal of the serum dilution giving half-maximal binding in the ELISA assay) in mice immunized on day 0, 14, 50 and 73 with Qβ-CETP1.

TABLE 7

| Qβ-CETP1 | ELISA Titers |
|---|---|
| 70 days after first immunizytion | 8512 |
| 80 days after first immunizytion | 19293 |

### Example 14

### Cloning, Expression and Purification of CETP1 Fused to the C-Terminus of AP205 VLP

### Cloning

The DNA fragment coding for the CETP1 peptide (SEQ ID NO:32) is created by annealing two complementary oligonucleotides encoding the peptide sequence of CETP1 and containing Kpn2I and Mph1103I restriction sites, respectively. The obtained fragment is digested with Kpn2I and Mph1103I and cloned in the same restriction sites into the vector pAP405-61 (as described in EXAMPLE 1 in of WO2006/032674) under the control of E. coli tryptophan operon promoter.

The protein AP205-11-CETP1 encoded by the resulting plasmid is: AP205 coat protein-GTAGGGSG-FGFPEHLLVDFLQSLS.

AP205-11-CETP1 is expressed and purified essentially as described in WO04/007538.

### Example 15

### Test of CETP Vaccines in the Cholesterol Fed Rabbit Model of Atherosclerosis

New Zealand White rabbits (n=12 per group) are vaccinated subcutaneously with 200 μg of VLP-CETP vaccine or VLP on day 0, and boosted on week 3, 6, 9, 12, 15, 19, 23 and 27. The rabbits are placed on a high cholesterol diet (0.25%) on week 16 and maintained on this diet for another 16 weeks. Plasma samples from fasted rabbits are collected at regular interval for antibody titer, lipoprotein, cholesterol and CETP activity measurements. The animals are sacrificed on week 32 and the aorta removed for atherosclerosis lesion analysis. The aorta are stained with oil red 0 after “en face” preparation of the Aorta, and the percentage of the aorta covered by lesions is calculated for each animal.

### Example 16

### Coupling of Bradykinin and des-Arg9-Bradykinin to Qβ VLP and Immunization of Mice

Bradykinin (BK) (SEQ ID NO:22) and des-Arg9-Bradykinin (SEQ ID NO:23) with a Cys fused to the N-terminus of both sequences or Bradykinin (BK) with a Cys fused to the C-terminus were chemically synthesized according to standard procedures. The peptides were coupled to Qβ VLP.

Adult female, C57BL/6 mice (10 per group) were vaccinated with either 50 μg Qβ-BK or Qβ-des-Arg9-BK coupled to Qβ subcutaneously (100 μl on two ventral sides) on days 0, 14 and 28. The vaccine was administered without adjuvant. Mice were bled retro-orbitally on day 0, 14, 21 and 30 and antibodies specific for BK or des-BK are measured by ELISA following standard protocol.

First, BK or des-Arg9-BK was coupled to RNase (SIGMA). Then ELISA plates were coated with Bradykinin peptides coupled to RNase at a concentration of 10 μg/ml in coating buffer (0.1 M NaHCO3, pH 9.6), over night at 4° C.

TABLE 8
Average anti-BK and anti-des-Arg9-BK specific IgG antibody titer
(expressed as a dilution factor) in mice immunized on day 0 and
14 with Qβ-BK or Qβ-des-Arg9-BK respectively.

| | Days after first immunization | | |
|---|---|---|---|
| Immunization | 14 | 21 | 30 |
| Qβ-C-BK | 3000 | 10000 | 3000 |
| Qβ-C-des-Arg9-BK | 20000 | 25000 | 15000 |
| PBS | 100 | 100 | 100 |

### Example 17

Efficacy of Vaccination Against Qβ-BK, Qβ-des-Arg9-BK for the Treatment of Collagen-Induced Arthritis

10 Male DBA/1 mice per group-were immunized intradermally three times (days 0, 14 and 28) with 50 μg of Qβ-BK, Qβ-des-Arg9-BK or Qβ alone. Then mice were injected twice intradermally (days 34 and 55) with 200 μg bovine type H collagen mixed with complete Freund's adjuvant.

After the second collagen/CFA injection mice are examined on a regular basis and a clinical score ranging from 0 to 3 is assigned to each limb according to the degree of reddening and swelling observed. Three weeks after the second collagen/CFA injection the average clinical score per limb is determined in the three experimental groups.

### Example 18

Efficacy of Vaccination Against Qβ-BK and Qβ-des-Arg9-BK for the Treatment of Allergic Airway Inflammation (AAI)

An experimental asthma model of allergic airway inflammation is used to assess the effects of vaccination against Bradykinin (BK) and des-Arg9-Bradykinin (des-Arg9-BK) on Th2-mediated immune responses characterized by: eosinophil influx into the lung, cytokine (IL-4, IL-5, IL-13) production, IgE antibody and mucous production and broncho hyper-responsiveness (BHR). Balb/c mice (5 per group) are immunised with either Qβ-BK or Qβ-des-Arg9-BK as described in EXAMPLE 16 or injected with Qβ alone. 35 days after the first immunisation, mice are injected intraperitonealy with 50 μg ovalbumin (OVA) in the presence or absence of adjuvant (Alhydrogel). 10 days later (i.e. day 45) all mice are daily intranasally challenged with 50 μg OVA in PBS on 4 consecutive days. 24 hours after the last challenge BHR is determined with a whole body phlegtismograph. Then mice are sacrificed at specific time points to analyze lung inflammation and Th2-mediated immune responses. Lung lavages are performed with PBS/1% BSA. The cells contained in the broncho alveolar lavage (BAL) are counted in a Coulter Counter (Instrumenten Gesellschaft AG) and differentiated with Maigrünwald-Giemsa staining as previously described (Trifilieff A, et al. Clin Exp Allergy. 2001 June; 31(6):934-42).

### Example 19

### Coupling gastrin or gastrin fragments to Qβ VLP

The following gastrin peptides were synthesized according to standard procedures.

G17 (1-9) C2:

(SEQ ID NO: 39)

pEGPWLEEEESSPPPPC
c1G17:

(SEQ ID NO: 40)

pEGPWLEEEEEAYGWMDFGGC
nG17amide:

(SEQ ID NO: 41)

CGGQGPWLEEEEEAYGWMDFCONH₂
nG17-G:

(SEQ ID NO: 40)

CGGQGPWLEEEEEAYGWMDFG
nG34amide:

(SEQ ID NO: 38)

CGGQLGPQGPPHLVADPSKKQGPWLEEEEEAYGWMDFCONH₂
nG34-G:

SEQ ID NO: 43)

CGGQLGPQGPPHLVADPSKKQGPWLEEEEEAYGWMDFG

The dialysed, derivatized Qβ VLP was subsequently used to couple c1G17. Briefly, 1 ml of derivatized Qβ VLP (at a concentration of 2 mg/ml) was reacted with 167 μl of a 10 mM peptide solution in DMSO and 100 μl of acetonitrile for 2 hours at 15° C. The coupled product was termed Qβ-c1 G17. The coupling efficiency [i.e. mol Qβ-gastrin/mol Qβ monomer (total)] was estimated, by densitometric analysis of the Coomassie blue stained SDS-PAGE, to be between 2.4 c1G17 fragments per Qβ monomer.

The dialysed, derivatized Qβ VLP was subsequently used to couple nG17amide, nG17-G, nG34amide or nG34-G. Briefly, 84 μl of derivatized Qβ VLP (at a concentration of 2 mg/ml) was reacted with 12 μl of a 10 mM peptide solution and 4 μl of H₂O 2O for 2 hours at 15°. The coupled products were termed Qβ-nG17amide, Qβ-nG17-G, Qβ-nG34amide and Qβ-nG34-G respectively.

### Example 20

### Coupling of G17(1-9)C2 (SEQ ID NO:39) to Diphtheria Toxoid (DT) and Qβ

The protocol used for coupling of G17(1-9)C2 to DT was similar to EXAMPLE 1 of U.S. Pat. No. 5,866,128. Briefly, DT (List Biological Laboratories) was activated by dissolving 1 mg of DT in 100 μl of 0.2 M sodium phosphate buffer, pH 6.6. Separately, 2 mg of SMPH was dissolved into 80 μl of DMSO. 12 μl of SMPH was added into 100 μl of DT. After 2 hours incubation at room temperature, the mixture was dialyzed twice for 2 hours against 2 L of 0.1 M sodium citrate buffer, pH 6.0. The coupled product was termed DT-G17(1-9)C2.

The dialysed, derivatized Qβ VLP was subsequently used to couple the G17(1-9)C2. Briefly, 84 μl of derivatized Qβ VLP was reacted with 6 μl of a 10 mM peptide solution in DMSO and 6 μl of H₂O for 2 hours at 18° C. The coupled product was termed Qβ-G17(1-9)C2.

### Example 21

### Immunization of Mice with Qβ-c1G17, Qβ-nG17amide, Qβ-nG17-G, Qβ-nG34amide, Qβ-nG34-G, Qβ-G17(1-9)C2 and DT-G17(1-9)C2

Adult female C5′7BL/6 mice were vaccinated with either Qβ-c1G17 (5 mice per group), Qβ-nG17amide, Qβ-nG17-G, Qβ-nG34amide and Qβ-nG34-G (3 mice per group). 50 μg of Qβ-c1G17 or 25 μg of Qβ-nG17amide, Qβ-nG17-G, Qβ-nG34amide and Qβ-nG34-G (obtained in EXAMPLE 24) were diluted in PBS to a volume of 200 μl and injected subcutaneously (100 μl on two ventral sides) on days 0 and 14. The vaccines were administered without adjuvant. As a control, a group of mice was injected with 50 μg of Qβ. Mice immunized with Qβ-C1G17 were bled retro-orbitally on day 0, 14, 21, 28, 42, 69, and 101 and mice which were immunized with Qβ-nG17amide, Qβ-nG17-G, Qβ-nG34amide and Qβ-nG34-G were bled retro-orbitally on day 0, 14, 21, 28, 42, 56, and 77.

Adult female C57/BL6 were immunized with Qβ-G17(1-9)C2 with 1 mg alum per mouse or without alum and DT-G17(1-9)C2 (5 mice per group) with 1 mg alum per mouse. 50 μg of Qβ-G17(1-9)C2 and DT-G17(1-9)C2 were diluted in PBS to a volume of 200 μl and injected subcutaneously (100 μl on two ventral sides) on days 0 and 14. Mice were bled retro-orbitally on day 0 and day 14. Titers of antibodies specific against these gastrin fragments were measured by ELISA by coating ELISA plates (96 well MAXIsorp, NUNC immuno plate) were coated with RNase-coupled c1G17 or nG17amide, nG17-G, nG34smide, nG34-G at a concentration of 10 μg/ml in coating buffer (0.1 M NaHCO₃, pH 9.6), over night at 4° C.

TABLE 9 Average anti-c1G17-, nG17amide, nG17-G, nG34amide or nG34-G-specific IgG antibody titer (expressed as a dilution factor) in mice immunized on day 0, and 14 with W-c1G17, Qβ-nG17amide, Qβ-nG17-G, Qβ-nG34amide and Qβ-nG34-G, respectively. This clearly demonstrates that a gastrin-VLP conjugate is able to induce a high antibody titer against gastrin fragments.

TABLE 9

Days after first immunization

| Immunization | 14 | 21 |
|---|---|---|
| Qβ-c1G17 | 6 358 | 19 694 |
| Qβ-nG17amide | 2 550 | 11 180 |
| Qβ-nG17-G | 447 | 11 874 |
| Qβ-nG34amide | 4 734 | 15 966 |
| Qβ-nG34-G | 2 343 | 53 942 |

TABLE 10 shows the average titers of G17(1-9)C2-specific antibodies. ELISA titers are expressed as serum dilutions which lead to half maximal OD in the ELISA assay. In mice immunized with Qβ-G17(1-9)C2 with or without Alum or DT-G17(1-9)C2, average titers of approximately 1:4242, 1:5838 and 1:788 respectively, were reached by day 14. The half maximal OD titer was less than 100, which was considered to be below the cut-off of the assay. This clearly demonstrates that Qβ-G17(1-9)C2 is able to induce earlier and higher antibody response than DT-G17(1-9)C2.

TABLE 10

| Immunization | 14 Days after first immunization |
|---|---|
| Qβ-G17(1-9)C2 without alum | 4242 |
| Qβ-G17(1-9)C2 with alum | 5838 |
| DT-G17(1-9)C2 with alum | 788 |

### Example 22

### Checking the Cross Reactivity of Sera which was Raised Against C1G17 to CCK8

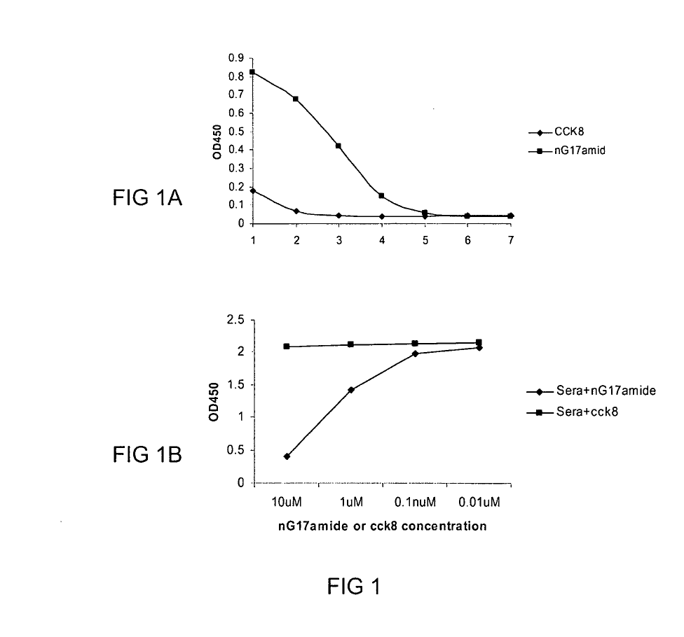

ELISA plates were coated with c1G17 or CCK8 (SIGMA) at a concentration of 0.2 mg/ml in coating buffer (0.1 M NaHCO3, pH 9.6), over night at 4° C. While ELISA titer from c1G17 coated plate was 1250, no clear reactivity to CCK was observed (FIG. 1A).

The cross activity was also checked in an inhibition ELISA. ELISA plates were coated with c1G17 or CCK8 (SIGMA) at a concentration of 0.2 mg/ml in coating buffer (0.1 M NaHCO3, pH 9.6), over night at 4° C. Mouse sera (14 days after immunization) raised against Qβ-c1G17 (obtained from EXAMPLE 21) were incubated with either serially diluted nG17amide or CCK8 at 37° C. for 2 hours on a heating block with 600 rpm shaking. Then these sera were added to the ELISA plate and incubated at RT for 2 h. While preincubation of nG17amide inhibited the recognition of sera to the coated nG17amide, no inhibition activity of CCK was observed. These two experiments showed that antibodies raised with Qβ-c1G17 did not cross react with CCK8 (FIG. 1B).

### Example 23

### Coupling C5a and C5a Fragment to Qβ

The murine C5a amino acid sequence containing an N-terminal CGSGG linker (SEQ 11) NO:47, hereafter named mC5acys) was chemically synthesized by Dictagene SA. The C-terminal 19 amino acids of the murine C5a sequence were chemically synthesized (EMC Microcollections) with an additional CGG linker at the N-terminus (SEQ ID NO:48, thereafter named mC5acys⁵⁹⁻⁷⁷).

A solution of 143 μM Qβ VLP in 20 mM HEPES, 150 mM NaCl, pH 7.2 was reacted with a 2-fold molar excess (286 μM) of (SMPH, Pierce) for 30 minutes at 25° C. with shaking. After dialysis, an equimolar amount of mC5acys was added to a 36 μM solution of SMPH-derivatized Qβ VLPs. Reaction volume was 100 μl and reactions were incubated for 2 hours at 15° C. with shaking.

A solution of 200 μm Qβ VLP in 20 mM HEPES, 150 mM NaCl, pH 7.2 was reacted with a 5-fold molar excess (1 mM) of SMPH (Pierce) for 30 minutes at 25° C. with shaking. After dialysis, a 5× molar excess of mC5acys⁵⁹⁻⁷⁷ was added to a 107 μM solution of SMPH-derivatized Qβ VLP. The reaction was incubated for 2 hours at 15° C. with shaking.

### Example 24

### Immunization of Mice with Qβ-mC5acys Vaccine and Detection of mC5acys-Specific Antibodies

Mice were immunized subcutaneously with 50 μs Qβ-mC5acys vaccine prepared as described in EXAMPLE 23 on days 0 and 14 and as required. Mice were bled retro-orbitally or via the tail vein at day 14 and day 21 and at subsequent timepoints. Serum was saved from these bleedings and analyzed by C5α-specific ELISA. Mice received 50 μg Qβ-VLP or received PBS only as negative controls. Anti-mC5acys IgG antibody titer was determined by ELISA by coating with 1 μg/ml mC5acys overnight in 0.1 M carbonate buffer (pH 9.6).

TABLE 11 shows representative results from this assay with sera from mice either immunized 24 days previously with Qβ-mC5acys, with Qβ VLP alone or left untreated. Mice that received the Qβ-mC5acys vaccine consistently showed an IgG antibody response against plate-coated mC5acys.

TABLE 11

| | Experimental Group Immunization | | |
|---|---|---|---|
| | Qβ-mC5acys | Qβ-VLP | PBS |
| Average anti- | 22410 | <50 | <50 |
| mC5acys IgG titer | | | |

Mice are immunized subcutaneously with 50 μg Qβ-mC5acys⁵⁹⁻⁷⁷ substantially the same as described above.

### Example 25

### Qβ-mC5acys Vaccine Immunization Neutralized the In Vivo Effects of Systemic mC5acys

The biological activity of mC5acys was determined in vivo in a neutropenia assay by measuring the apparent drop in blood granulocyte numbers after the intravenous administration of small quantities of mC5acys.

Female C57BL/6 mice (6-8 weeks of age) were anesthetised and injected with 100 μl solution via the lateral tail vein. The mice received either PBS, mC5acys in PBS or Qβ capsid in PBS. After three minutes the mice were bled via the retro-orbital route and 100 μl of whole blood transferred to 2 ml PBS containing the anti-coagulant heparin (Roche). Cells were pelleted by centrifugation at 450 xg for 10 minutes at room temperature. After aspirating the supernatant, the cell pellet was resuspended in 2 ml Tris Ammonium Chloride (TAC) solution (17 mM Tris, 126 mM NH₄Cl, pH 7.2) for 5 minutes at room temperature to lyse the red blood cells. The remaining cells were pelleted by centrifugation and the TAC treatment repeated. The remaining cells were re-pelleted by centrifugation and resuspended in 50 μl flow cytometry wash buffer (Dulbecco's PBS containing 2% (v/v) fetal bovine serum and 0.1% NaN₃). Cells were passed though a flow cytometer (FACSCalibur, Becton Dickenson) and the fraction of granulocytes determined by forward and side light scatter gating.

A representative experiment demonstrating that mCa5cys induces neutropenia is given in TABLE 6. In this case 1 nmol mC5acys induces statistically significant neutropenia compared to PBS treated mice and mice that received 1 μg Qβ capsid protein, showing that the synthesized mC5acy has biological activity.

C57BL/6 mice were immunized subcutaneously on the flank with 50 pg Qβ-mC5acys diluted in Dulbecco's PBS. Control mice received Qβ alone or were untreated. Immunizations were performed on day 0 and day 14 of the experiment. On day 22 after the first immunization 50 pmol mC5acys was injected intravenously via the lateral tail vein to induce systemic neutropenia. In mice immunized with Qβ VLP alone or in untreated mice there is a drop in the percentage of granulocytes in the blood 3 minutes after the injection of 50 pmol mC5acys. In mice vaccinated with Qβ-mC5acys this decrease in the percentage of blood granulocytes is prevented. Thus anti-mC5a antibodies raised in mice by immunization with Qβ-mC5acys are able to neutralize the systemic neutropenia response induced by the administration of intravenous mC5acys (TABLE 12).

TABLE 12

| | | Percentage granulocytes in | |
|---|---|---|---|
| | Substance injected | retro-orbital blood sample 3 | |
| Experimental Treatment | intravenously | minutes after i.v. injection | ±SD |
| C57BL/6, unimmunized | PBS | 10.5 | 1.8 |
| C57BL/6, unimmunized | 70 pmol Qβ-VLP | 10.0 | 0.9 |
| C57BL/6, unimmunized | 1 nmol mC5acys | 3.8 | 1.9 |
| C57BL/6, Qβ-mC5acys | 50 pmol mC5acys | 9.1 | 1.0 |
| immunized | | | |
| C57BL/6, Qβ-VLP | 50 pmol mC5acys | 4.4 | 0.4 |
| immunized | | | |
| C57BL/6, PBS treated | 50 pmol mC5acys | 4.7 | 1.1 |

### Example 26

### Immunization with Qβ-mC5Acys VLP Alleviates Disease in a Collagen-Induced Arthritis Model in Mice

Male 6 week old DBA/1JCr1 mice (Charles River, Deutschland) were immunized subcutaneously on the flanks with either 50 μg Qβ-mC5acys (n=8) or 50 μg Qβ VLP (n=8), both diluted in Dulbecco's PBS. Two further booster immunizations of either 30 μg Qβ-mC5a or 30 μg Qβ VLP were also given subcutaneously, on days 15 and 24 after the initial immunization. Mice were immunized intradermally at the base of the tail twice on days 35 and 57 after the initial immunization with 100 μg bovine type II collagen (MD Biosciences) emulsified using glass syringes as a 1:1 ratio in Complete Freund's Adjuvant (CFA). CFA was prepared from Incomplete Freund's Adjuvant (Difco Laboratories) containing 5 mg/ml heat-killed Mycobacterium tuberculosis strain H37RA (Difco Laboratories). The mice were then monitored for the induction and severity of collagen-induced arthritis by daily measurements of fore and hind limb joint thickness and by the daily estimation of joint clinical scores. Joint thickness was measured using constant-tension calipers. Clinical scores were assigned on the basis of the following scale: Score 0—no swelling, joint normal; Score 1—mild redness and/or swelling of the digits/paws; Score 2—Redness and swelling, involving the entire paw/joint; Score 3—Severe swelling, deformation of the paws/joints with ankylosis. Experimental observations were continued until day 15 after the final collagen/CFA injection (day 72 after the initial immunizations

TABLE 13 shows the average increase in joint thickness across all limbs after the final collagen/CFA injection. The average increase in joint thickness is lower on most days for the Qβ-mC5acys vaccinated group compared to the Qβ control, with this difference having a p value <0.1 (by 2-tailed student's t-test) on days 5, 7 and 10 after the final collagen/CFA injections.

TABLE 13

| Time (days) | Average percentage increase in limb | | | |
|---|---|---|---|---|
| after last | thickness (all limbs), baseline is 100% | | | |
| collagen | Qβ-VLP | | Qβ-mC5acys | |
| injection | immunized (n = 8) | ±SD | immunized (n = 8) | ±SD |
| 2 | 100 | — | 100 | — |
| 3 | 102.5 | 5.5 | 99.5 | 7.1 |
| 5 | 103.2 | 7.4 | 104.3 | 8.3 |
| 6 | 103.7 | 5.9 | 102.0 | 11.9 |
| 7 | 111.4 | 7.2 | 104.6 | 19.6 |
| 8 | 110.3 | 7.7 | 106.4 | 14.4 |
| 9 | 111.4 | 12.9 | 110.0 | 15.0 |
| 10 | 115.8 | 13.7 | 108.7 | 16.7 |
| 12 | 120.1 | 18.3 | 112.5 | 23.6 |
| 14 | 122.7 | 23.1 | 114.3 | 24.6 |
| 15 | 125.9 | 24.2 | 118.0 | 23.6 |

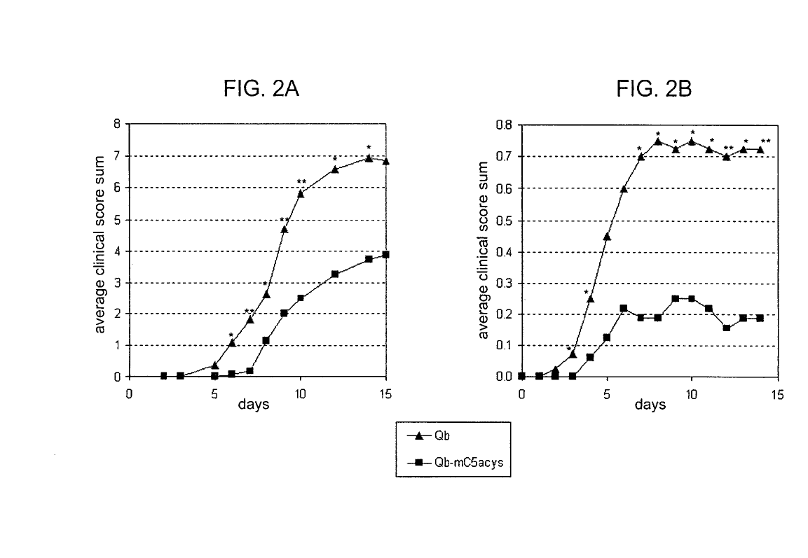

FIG. 2a shows the average clinical score sum across all limbs after the final collagen/CFA injection. The average clinical score sum is consistently lower in the Qβ-mC5acys vaccinated group compared to the Qβ VLP control, with this difference having a p value <0.1 (by 2-tailed student's t-test) on days 6, 8 12 and 14 and a p value <0.05 (by 2-tailed student's West) on days 7, 9 and 10 after the final collagen/CFA injection. This result implies that vaccination with Qβ-mC5acys reduces the severity of collagen-induced arthritis in mice when compared to Qβ carrier vaccinated animals.

### Example 27

### Immunization with Qβ-mC5Acys VLP Alleviates Disease in an Anti-Collagen-Monoclonal Antibody-Cocktail Induced Arthritis Model in Mice

Female 6-8 week old balb/c mice (Charles River) were immunized subcutaneously on the flanks with either 50 μg Qβ-mC5acys (n=5) or 50 μg Qβ VLP (n=5), all diluted in Dulbecco's PBS. Two further booster immunizations of either 50 μg Qβ-mC5a or 50 μs Qβ VLP were also given subcutaneously, on days 21 and 35 after the initial immunization. Mice were immunized intravenously on day 41 after the initial immunization with 200 μl anti-collagen monoclonal antibody cocktail (MDBiosciences) followed by intraperitoneal injection of 100 ul LPS solution (MDBiosciences) 1 day later. The mice were then monitored for the induction and severity of anti-collagen monoclonal antibody induced-arthritis substantially the same as described in EXAMPLE 26. Experimental observations were continued until day 14 after the anti-collagen monoclonal antibody cocktail injection (day 55 after the initial immunizations).

FIG. 2b shows the average clinical score sum across all limbs after the anti-collagen-monoclonal antibody-cocktail injection. The average clinical score sum is consistently lower in the Qβ-mC5acys vaccinated group compared to the Qβ VLP control, with this difference having a p value <0.1 (by 2-tailed student's t-test) on days 3, 4, 7, 8, 9, 10, 11 and 13 and a p value <0.05 (by 2-tailed student's t-test) on days 12 and 14 after the final collagen/CFA injection. This result implies that vaccination with Qβ-mC5acys reduces the severity of anti-collagen-monoclonal antibody-induced arthritis in mice when compared to Qβ carrier vaccinated animals.

### Example 28

### Immunization with Qβ-mC5Acys VLP and the New Zealand Black/New Zealand White F1 Cross Model of Systemic Lupus Erythematosus

NZB/NZW F1 mice spontaneously develop an autoimmune disease with striking similarities to human systemic lupus erythematosus (Andrews et. al. J. Exp. Med., 148: 1198, 1978). Female 16 week old NZB/NZW F1 mice (Charles River) were immunized subcutaneously on the flanks with either 50 μg Qβ-mC5acys (n=20) or 50 μg Qβ VLP (n=20), all diluted in Dulbecco's PBS. Two further booster immunizations of either 50 μg Qβ-mC5a or 50 μg Qβ VLP were also given subcutaneously, on days 14 and 28 after the initial immunization. A further booster of either 50 μg Qβ-mC5a or 50 μg Qβ VLP in alum was given on day 58. The amount of protein excreted in the urine (proteinuria) was measured weekly from 16 (day 0) till 29 weeks of age (day 91) by colourometric analysis using dipsticks (Roche). Proteinuria is further measured weekly till 52 weeks of age and antibody titres are kept high by further boosting as required.

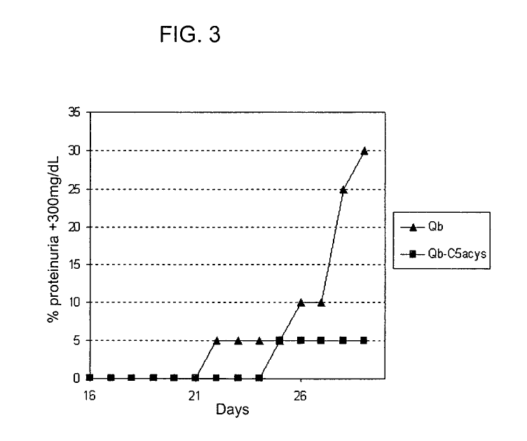

FIG. 3 shows the percentage of mice whose proteinuria reading has reached 300 mg/dL. These data show that 30% of mice in the Qβ treated group had proteinuria readings of greater than 300 μg/ml by the age of 29 weeks. In comparison only one mouse in the QβC5acys treated group had a reading above 300 μg/ml at this age. This particular mouse had low C5acys antibody titres as determined by ELISA. This result implies that vaccination with Qβ-mC5acys reduces the incidence or delays the onset of proteinuria in the New Zealand black/New Zealand white F1 model of systemic lupus erythematosus compared to Qβ carrier vaccinated animals.

What is claimed is:

1. A composition comprising:
(a) a virus-like particle (VLP) with at least one first attachment site; and
(b) at least one antigen with at least one second attachment site,
wherein said at least one antigen is an antigen of the invention selected from the group consisting of:
a) CCR5 of the invention;
b) C5a of the invention;
c) CXCR4 of the invention;
d) Gastrin of the invention; and
e) CETP of the invention;
and wherein (a) and (b) are linked through said at least one first and said at least one second attachment site.

2. The composition of claim 1 comprising:
(a) a virus-like particle of an RNA-bacteriophage with at least two first attachment sites; and
(b) at least one CCR5 extracellular domain PNt with at least two second attachment sites; wherein said CCR5 extracellular domain PNt comprises:
(i) a Nta domain or a Nta domain fragment, and
(ii) a Ntb domain comprising amino acids 23 to 27 of SEQ ID NO:27 (SEQ ID NO:56) or Ntb domain fragment comprising amino acids 23 to 27 of SEQ ID NO:27, and
wherein the first or the second of said at least two second attachment sites comprises a sulfhydryl group, and
wherein the first of said at least two second attachment sites is located upstream of the N-terminus of said amino acids 23 to 27 of SEQ ID NO:27; and
wherein the second of said at least two second attachment sites is located downstream of the C terminus of said CCR5 extracellular domain PNt; and
wherein said VLP of said RNA-bacteriophage and said CCR5 extracellular domain PNt are linked by at least one non-peptide covalent bond.

3. The composition of claim 2, wherein said CCR5 extracellular domain PNt with at least two second attachment sites does not comprise a further sulfhydryl group besides said two sulfhydryl groups comprised by said first and said second of said at least two second attachment sites.

4. The composition of claim 2 or 3, wherein the first of said at least two second attachment sites corresponds to the sulfhydryl group of the cysteine residue of SEQ ID NO:27.

5. The composition of any one of the claims 2-4, wherein said CCR5 extracellular domain PNt comprises the amino acid sequence of SEQ ID NO:27.

6. The composition of any one of the claims 2-5 further comprising a linker, wherein said linker is fused to the C-terminus of said CCR5 extracellular domain PNt, and wherein said linker comprises said second of said at least two second attachment sites, wherein preferably said linker is a cysteine or an amidated cysteine.

7. The composition of any one of the claims 2-6, wherein said first and said second of said at least two second attachment sites associate with said at least two first attachment sites through at least two non-peptide covalent bonds.

8. The composition of any one of the claims 2-7, wherein said RNA-bacteriophage is Qβ or AP205.

9. The composition of any one of the claims 2-8, wherein each of said at least two first attachment sites comprises an amino group.

10. The composition of claim 1, wherein said CCR5 of the invention is a CCR5 extracellular domain, preferably said CCR5 extracellular domain is CCR5 extracellular domain PNt, further preferably said PNt domain comprises the amino acid sequence as of SEQ ID NO:27.

11. The composition of claim 1, wherein said CCR5 of the invention is a CCR5 extracellular domain fragment, preferably said CCR5 extracellular domain fragment is CCR5 extracellular domain ECL2A fragment, further preferably said CCR5 extracellular domain ECL2 fragment comprises an amino acid sequence selected from the group consisting of:
(a) SEQ ID NO:25; and
(b) SEQ ID NO:26.

12. The composition of claim 1, wherein said gastrin of the invention comprises, consists essentially of, or alternatively consists of an amino acid sequence selected from the group consisting of
a) SEQ ID NO:33
b) SEQ ID NO:34;
c) SEQ ID NO:35;
d) SEQ ID NO:36;
e) SEQ ID NO:37;

13. The composition of claim 1, wherein said C5a of the invention is a C5a protein, preferably said C5a protein comprises, consists essentially of, or alternatively consists of an amino acid sequence selected from a group consisting of:
(a) SEQ ID NO:45; and
(b) a polypeptide derived from SEQ ID NO:45, in which three, preferably two, preferably one amino acid of SEQ ID NO:45 has been modified by insertion, deletion and/or substitution.

14. The composition of claim 1 or any one of the claims 10-13, wherein said VLP is of an RNA-bacteriophage.

15. The composition of claim 14, wherein said RNA-bacteriophage is Qβ, fr, GA or AP205.

16. The composition of claim 1 or any one of the claims 10-15, wherein said VLP with first attachment site is linked to said antigen of the invention with second attachment site via at least one covalent bond, wherein preferably said covalent bond is a peptide bond, wherein said VLP is of an RNA bacteriophage AP205.

17. The composition of claim 1 or any one of the claims 10-15, wherein said first attachment site is linked to said second attachment site via at least one covalent bond, wherein preferably said covalent bond is a non-peptide bond.

18. The composition of any one of the preceeding claims, wherein said first attachment site comprises, preferably an amino group of a lysine.

19. The composition of any one of the preceeding claims, wherein said second attachment site comprises a sulthydryl group, preferably a sulfhydryl group of a cysteine.

20. A vaccine, comprising the composition of any one of the claims 1-19, wherein preferably said vaccine is devoid of an adjuvant.

21. A pharmaceutical composition comprising:
(a) the composition of any one of the claims 1-19 or the vaccine of claim 20; and
(b) an acceptable pharmaceutical carrier.

22. A method of producing the composition of claim 1 or any one of the claims 10-19, or the vaccine of claim 20, comprising:
(a) providing a VLP with at least one first attachment site;
(b) providing at least one antigen of the invention with at least one second attachment site; and
(c) linking said VLP and said at least one antigen of the invention to produce said composition, wherein said at least one antigen of the invention and said VLP are linked through said at least one first and said at least one second attachment site.

23. Use of the composition of claim 2-11 for the manufacture of a medicament for the treatment of AIDS.

24. Use of the composition of claim 12 for the manufacture of a medicament for the treatment of gastrointestinal cancer.

25. Use of the composition of any of the claim 13 for the manufacture of a medicament for the treatment of arthritis.
